(19) United States
(12) Patent Application Publication
(10) Pub. No.: US 2023/0212635 A1
(43) Pub. Date: Jul. 6, 2023

(54) METHODS OF PRODUCING RECOMBINANT COMPLEMENT PROTEINS, VECTORS AND THERAPEUTIC USES THEREOF

(71) Applicant: Gyroscope Therapeutics Limited, London (GB)

(72) Inventors: Kevin Marchbank, North Shields (GB); David Kavanagh, Newcastle upon Tyne (GB); Claire Harris, Newcastle upon Tyne (GB); Thomas Cox, Newcastle upon Tyne (GB)

(73) Assignee: Gyroscope Therapeutics Limited, London (GB)

(21) Appl. No.: 17/928,798

(22) PCT Filed: Nov. 19, 2021
(86) PCT No.: PCT/GB2021/053009

§ 371 Date: Nov. 30, 2022

(30) Foreign Application Priority Data
Nov. 20, 2020 (GB) 2018320.8

(51) Int. Cl. C12P 21/02; C12N 15/86; C12N 9/50; C12N 5/071

(52) U.S. Cl. CPC C12P 21/02; C12N 15/86; C12N 9/50; C12Y 304/21075; C12N 5/0687; C12N 2840/203; C12N 2750/14143

(57) ABSTRACT

Aspects of the present invention relate to the recombinant production of a mature complement system protein. Certain embodiments of the present invention relate to recombinant production of fully mature human complement Factor I protein (CFI). Included herein are details of an expression vector with which to recombinantly express fully mature human CFI from mammalian cells. Further disclosed are chromatography steps with which to purify recombinantly expressed CFI. Certain aspects of the present invention relate to the use of an expression system in gene therapy and the like. Certain embodiments of the present invention relate to use of said vector as a medicament, for example for use in the treatment of complement-mediated disorders.

### TECHNICAL FIELD

Aspects of the present invention relate to the recombinant production of a mature complement system protein. Certain embodiments of the present invention relate to recombinant production of fully mature human complement Factor I protein (CFI). Included herein are details of an expression vector with which to recombinantly express fully mature human CFI from mammalian cells. Further disclosed are chromatography steps with which to purify recombinantly expressed CFI. Certain aspects of the present invention relate to the use of an expression system in gene therapy and the like. Certain embodiments of the present invention relate to use of said vector as a medicament, for example for use in the treatment of complement-mediated disorders.

### BACKGROUND TO THE INVENTION

The complement system is a part of the innate immune system which is made up of a large number of discrete plasma proteins that react with one another to opsonize pathogens and induce a series of inflammatory responses that help to fight infection. Many complement proteins exist in a ‘precursor’ form and are activated at the site of inflammation. In addition to protecting the host against invading pathogens, it bridges innate and adaptive immunity and disposes of immune complexes and injured tissues and cells. The alternative pathway of complement (AP) is continually activated by a tick-over mechanism and can also be triggered by the classical and lectin pathways. In the AP, complement component 3 (C3) undergoes spontaneous hydrolysis, depositing C3b onto the surface of foreign and host cells in the vicinity. On an activating surface such as a bacterium, C3b joins with Factor B, which then is cleaved by Factor D to form the C3 convertase, C3bBb. The binding of properdin stabilises this enzyme. This enzyme complex then cleaves more C3 to C3b to initiate a feedback loop. Downstream of this amplification loop, C3b may also join with the C3 convertase to form the C5 convertase, C3bBbC3b. C5 is cleaved to the anaphylatoxin C5a and C5b, which initiates formation of the lytic membrane attack complex (C5b-9).

To protect host cells from collateral complement damage, many soluble and membrane-associated complement regulatory proteins function to inactivate complement on their surfaces. Complement factor I (CFI) is an 88 kDa serum glycoprotein which is the key regulatory enzyme of the complement system. It is a serine protease that cleaves the a chains of C3b and C4b, but only in the presence of its cofactor proteins: factor H (FH) for C3b cleavage, C4 binding protein (C4BP) for C4b cleavage and CD46 and complement receptor 1 for both.

It is expressed in numerous tissues but principally by liver hepatocytes. The encoded proprotein (pro-CFI) is cleaved to produce both heavy and light chains, which are linked by disulphide bonds to form a heterodimeric glycoprotein. This heterodimer can cleave and inactivate the complement components C4b and C3b, preventing the assembly of the C3 and C5 convertase enzymes. Defects in this gene cause CFI deficiency, an autosomal recessive disease associated with a susceptibility to pyogenic infections.

Dysregulation of the complement system is known to mediate several disorders. Rare genetic variants in the CFI gene have been associated with a predisposition to atypical hemolytic uremic syndrome, a disease characterized by acute renal failure, microangiopathic hemolytic anemia and thrombocytopenia. Recently, low levels of circulating CFI have been identified in individuals with rare genetic variants in the CFI gene and these are associated with advanced Age-Related Macular Degeneration (AMD) supporting the role of CFI in risk of AMD (Kavanagh et al., 2015, Hallam et al 2020). AMD is the most common cause of vision loss in individuals over the age of 50 and currently there are few treatment options. CFI mediated complement system regulation has also been implicated in the progression of early phase Alzheimer's disease (Hakobyan et al, 2016). CFI deficiency has also been recently associated with fulminant cerebral inflammation (Altmann et al, 2020). This research suggests that enhancing CFI activity in these individuals may have some therapeutic benefit.

As mentioned above, when CFI is produced, it is initially synthesized as a single chain precursor (pro-CFI) in which a four-residue linker peptide (RRKR) connects the heavy chain to the light chain. The pro-CFI is inactive. During processing, the linker is cleaved by a calcium-dependent serine endoprotease known as Furin, leaving the heavy and light chain of mature CFI held together by a single disulphide bond.

Currently, efforts to produce compositions comprising a high percentage of recombinant mature CFI are either ineffectual or require multiple highly optimized downstream processing steps. Typically, prior art methods result in incomplete cleavage of the precursor to the mature CFI protein which results in compositions comprising significant amounts of uncleaved precursor protein in the first instance which requires downstream cleavage steps. Furthermore, previous efforts have resulted in compositions which have reduced activity as compared to plasma-derived Complement Factor I.

Wong et al showed that co-transfection of COS-1 cells with two distinct vectors independently encoding human pro-CFI and furin only resulted in around synthesis of 50% mature CFI. Furthermore, the independent pro-CFI and furin vector design generally precludes the use of such an expression system in gene therapy due to the requirement, and unlikely event, of co-transfection of cells within perfuse tissue.

WO2018/170152 A1 discloses a method for recombinant expression of pro-CFI from a pDR2 eukaryotic expression vector in Chinese Hamster Ovary (CHO) cells, followed by in vitro incubation of purified recombinant pro-CFI with Furin to produce mature CFI. The subsequent cleavage of purified recombinant pro-CFI with Furin may be considered to add time and cost to the production procedure. The process can also only be conducted in vitro, and as such cannot be used in vivo as a gene therapy.

It is therefore an aim of certain embodiments of the present invention to mitigate the problems associated with the prior art.

It is an aim of certain embodiments to provide a method for recombinant production of a mature complement system protein.

It is an aim of certain embodiments to provide a method for recombinant production of mature Complement Factor I in vivo.

It is an aim of certain embodiments to provide an expression vector capable of expressing a mature Complement Factor I in vivo.

It is an aim of certain embodiments to provide a method for recombinant production of mature Complement Factor I within a subject.

It is an aim of certain embodiments to provide an expression vector comprising a) a nucleic acid molecule encoding a precursor Complement Factor I protein or variant thereof; and b) a nucleic acid molecule encoding a furin protein or variant thereof wherein said furin protein or variant thereof is capable of cleaving the encoded precursor Complement Factor I protein to produce a mature Complement Factor I protein, wherein the expression vector increases the concentration of mature Complement Factor I within a human subject.

It is an aim of certain embodiments to provide an expression vector for use in the treatment of a complement mediated disorder, wherein the expression vector comprises a) a nucleic acid molecule encoding a precursor Complement Factor I protein or variant thereof; and b) a nucleic acid molecule encoding a furin protein or variant thereof wherein said furin protein or variant thereof is capable of cleaving the encoded precursor Complement Factor I protein to produce a mature Complement Factor I protein.

It is an aim of certain embodiments to provide a method of treating a complement mediated disorder in a subject, comprising administering to the subject in need thereof an expression vector comprising a) a nucleic acid molecule encoding a precursor Complement Factor I protein or variant thereof; and b) a nucleic acid molecule encoding a furin protein or variant thereof wherein said furin protein or variant thereof is capable of cleaving the encoded precursor Complement Factor I protein to produce a mature Complement Factor I protein.

It is an aim of certain embodiments of the present invention to provide a method for producing a high concentration of recombinantly produced mature Complement Factor I.

It is an aim of certain embodiments of the present invention to provide a Complement Factor I protein for use in the treatment of a complement associated disorders.

### SUMMARY OF THE DISCLOSURE

In a first aspect of the present invention there is provided an expression vector for the production of a mature recombinant Complement Factor I protein or variant thereof, wherein the expression vector comprises:
a. a nucleic acid molecule encoding a precursor Complement Factor I protein or variant thereof; and
b. a nucleic acid molecule encoding a furin protein or variant thereof wherein said furin protein or variant thereof is capable of cleaving the encoded precursor Complement Factor I protein to produce a mature Complement Factor I protein.

In certain embodiments, the furin protein is capable of cleaving greater than 50% of the recombinant precursor Complement Factor I protein or variant thereof.

In certain embodiments, the expressed recombinant furin protein or variant thereof is capable of cleaving greater than 55%, e.g. 60%, 65%, 70%, 75%, 80%, 85%, 90% or greater than 95% of the recombinant precursor Complement Factor I protein or variant thereof.

In certain embodiments, the expression vector is suitable for in vivo use. Further details of the expression vector are provided herein.

In a further aspect of the present invention there is provided an expression system comprising a vector for the production of a mature recombinant Complement Factor I protein or variant thereof, wherein the vector comprises:
a. a nucleic acid molecule encoding a precursor Complement Factor I protein or variant thereof; and
b. a nucleic acid molecule encoding a furin protein or variant thereof wherein said furin protein is capable of cleaving the encoded precursor Complement Factor I protein to produce a mature Complement Factor I protein.

In certain embodiments, the furin protein is capable of cleaving greater than 50% of the recombinant precursor Complement Factor I protein or variant thereof.

In certain embodiments, the expressed recombinant furin protein or variant thereof is capable of cleaving greater than 55%, e.g. 60%, 65%, 70%, 75%, 80%, 85%, 90% or greater than 95% of the recombinant precursor Complement Factor I protein or variant thereof.

In certain embodiments, the expression vector comprises a promoter element.

In certain embodiments, the promoter element is adapted for in vivo use.

In certain embodiments, the promoter element is upstream of the nucleic acid molecule encoding a precursor complement Factor I protein or variant thereof. In certain embodiments, the promoter element is upstream of the nucleic acid molecule encoding a furin protein.

In certain embodiments, the promoter element is upstream of the nucleic acid molecule encoding a precursor complement Factor I protein or variant thereof and nucleic acid molecule encoding a furin protein or variant thereof.

In certain embodiments, the promoter element is differentially activated in response to environmental changes of the expression system.

In certain embodiments, the expression vector comprises a nucleic acid molecule encoding a translation initiation sequence.

In certain embodiments, the nucleic acid molecule encoding a translation initiation sequence is positioned downstream of the promoter element and upstream of the nucleic acid molecule encoding the precursor Complement Factor I protein or variant thereof and the nucleic acid molecule encoding the furin protein or variant thereof.

In certain embodiments, the nucleic acid molecule encoding a translation initiation sequence comprises a sequence as set forth in SEQ. ID. No. 7.

In certain embodiments, the expression vector further comprises a nucleic acid molecule encoding an internal ribosome entry site.

In certain embodiments, the nucleic acid molecule encoding the internal ribosome entry site is positioned between the nucleic acid molecule encoding the precursor Complement Factor I protein or variant thereof and the nucleic acid molecule encoding the furin protein.

In certain embodiments, the nucleic acid molecule encoding the furin protein is upstream of the IRES and the nucleic acid molecule encoding the Complement Factor I protein is downstream of the IRES.

In certain embodiments, the internal ribosome entry site is an encephalomyocarditis virus IRES.

In certain embodiments, the nucleic acid molecule encoding the internal ribosome entry site comprises a nucleic acid sequence as set forth in SEQ. ID. 9.

Other strategies that may be used for multigene co-expression include use of multiple promoters in a single vector, proteolytic cleavage sites between genes, and “self-cleaving” 2A peptides.

In certain embodiments, the expression vector may comprise a nucleic acid molecule encoding a proteolytic cleavage site.

In certain embodiments, the expression vector may comprise a nucleic acid molecule encoding a self-cleaving peptide, for example a 2A self-cleaving peptide, for example T2A, P2A, E2A or F2A.

Preferably the nucleic acid molecule encoding the self-cleaving peptide is positioned between the nucleic acid molecule encoding the furin protein and the nucleic acid molecule encoding the precursor Complement Factor I protein. In certain embodiments, the nucleic acid molecule encoding precursor Complement Factor I protein is upstream of the nucleic acid molecule encoding furin protein, wherein the nucleic acid molecule encoding the self-cleaving peptide is positioned between the nucleic acid molecules encoding precursor Complement Factor I and furin protein. In certain embodiments, the nucleic acid molecule encoding precursor Complement Factor I is downstream of the nucleic acid molecule encoding furin protein, wherein the nucleic acid molecule encoding the self-cleaving peptide is positioned between the nucleic acid molecules encoding precursor Complement Factor I and furin protein. Preferably, the expression vector comprises a nucleic acid molecule encoding an internal ribosome entry site.

In certain embodiments, the recombinant precursor Complement Factor I is a mammalian Complement Factor I protein. In certain embodiments, the mammalian precursor Complement Factor I is a human precursor Complement Factor I protein.

In certain embodiments, the mature human Complement Factor I protein comprises a first amino acid sequence selected from an amino acid sequence as set forth in SEQ. ID. No. 2 (heavy chain) or an amino acid sequence having at least 70%, 80% or 85% sequence identity to the amino acid sequence as set forth in SEQ. ID. No. 2, and a second amino acid sequence as set forth in SEQ. ID. 3 (light chain) or an amino acid sequence having at least 85% sequence identity to the amino acid sequence set forth in SEQ. ID. No. 3, wherein the first and second amino acid sequences are linked via a disulphide bond.

In certain embodiments, the precursor human Complement Factor I comprises an amino acid sequence as set forth in SEQ. ID. No. 1, or an amino acid sequence having at least 90% sequence identity to the amino acid sequence as set forth in SEQ. ID. No. 1, e.g. 91, 92, 93, 94, 95, 96, 97, 98 or 99% sequence identity.

In certain embodiments, the first amino acid sequence and/or the second amino acid sequence possess one or more conservative amino acid substitutions.

In certain embodiments, the expressed complement factor I protein is glycosylated, acetylated, phosphorylated, pegylated, and/or ubiquitinated.

In certain embodiments, the encoded furin protein or variant thereof is a mammalian furin protein or variant thereof. In certain embodiments, the encoded furin protein or variant thereof is a human furin protein or variant thereof.

In certain embodiments, the human furin protein comprises an amino acid sequence selected 30 from an amino acid sequence as set forth in SEQ. ID. No. 5 or an amino acid sequence having at least 70%, 75%, 80% or 85% sequence identity to the amino acid sequence set forth in SEQ. ID. No 5.

In certain embodiments, the human furin protein comprises an amino acid sequence having 35 at least 90% sequence identity to the amino acid sequence as set forth in SEQ. ID. No: 5, e.g. 91, 92, 93, 94, 95, 96, 97, 98 or 99% sequence identity.

In certain embodiments, the human furin protein comprises an amino acid sequence having one or more conservative amino acid substitutions as compared to the amino acid sequence as set forth in SEQ. ID. No 5.

In certain embodiments, the vector comprises a nucleic acid sequence selected from the nucleic acid sequence as set forth in SEQ. ID. No. 10 or a nucleic acid sequence having at least 75%, 80% or 85% sequence identity to the nucleic acid sequence as set forth in SEQ. ID. No. 10.

In certain embodiments, the vector comprises a nucleic acid sequence having at least 75%, 80%, 85% or 90% sequence identity to the nucleic acid sequence as set forth in SEQ. ID. No. 8, e.g. 91, 92, 93, 94, 95, 96, 97, 98 or 99% sequence identity.

In certain embodiments, the expression vector comprises at least one nucleic acid molecule encoding a resistance marker.

In certain embodiments, the nucleic acid molecule encoding a resistance marker encodes a hygromycin resistance marker.

In certain embodiments, the nucleic acid molecule encoding a hygromycin resistance marker comprises a nucleic acid sequence as set forth in SEQ. ID. No 13.

In certain embodiments, the nucleic acid molecule encoding a resistance marker encodes an ampicillin resistance marker.

In certain embodiments, the nucleic acid molecule encoding an ampicillin resistance marker comprises a nucleic acid sequence as set forth in SEQ. ID. No. 16.

In certain embodiments, the expression system further comprises a host cell, wherein the host cell is a eukaryotic cell, wherein optionally the eukaryotic cell is a Human Embryonic Kidney 293T.

In certain embodiments, the expression system is adapted for in vivo expression.

In certain embodiments, the expression system is adapted for in vitro expression.

Aptly, the expression vector is a viral vector. In certain embodiments, the viral vector is a non-integrating viral vector. The non-integrating viral vector may be an adenovirus vector.

In certain embodiments, the viral vector is an integrating viral vector.

Aptly, the integrating viral vector may be an adeno-associated virus vector.

In certain embodiments, the AAV vector is in the form of an AAV vector particle.

In certain embodiment, the AAV vector particle comprises an AAV2 genome and AAV2 capsid proteins, an AAV2 genome and AAV5 capsid proteins, or an AAV2 genome and AAV8 capsid proteins.

In a further aspect of the present invention, there is provided a method for producing a recombinant mature Complement Factor I protein or variant thereof, the method comprising:
a. expressing (i) a recombinant precursor Complement Factor I protein or variant thereof and (ii) a recombinant furin protein or variant thereof in a host cell under conditions suitable for the expressed furin protein to cleave the expressed recombinant precursor Complement Factor I protein or variant thereof to form the recombinant mature Complement Factor I protein or variant thereof, wherein optionally greater than 50% of the recombinant precursor Complement Factor I protein or variant thereof is cleaved by the expressed furin protein.

In certain embodiments, greater than 55%, e.g. 60%, 65%, 70%, 75%, 80%, 85%, 90% or greater than 95% of the expressed recombinant precursor Complement Factor I protein or variant thereof is cleaved by the expressed recombinant furin protein.

In certain embodiments, the method comprises transfecting a host cell with an expression vector which comprise a nucleic acid molecule encoding the precursor Complement Factor I protein or variant thereof and a nucleic acid molecule encoding the furin protein.

In certain embodiments, the expression vector further comprises:
a. a nucleic acid molecule encoding an internal ribosome entry site, wherein the nucleic acid molecule encoding the internal ribosome entry site is positioned between the nucleic acid molecule encoding the precursor Complement Factor I protein or variant thereof and the nucleic acid molecule encoding the furin protein or variant thereof;
b. a promoter element, wherein the promoter element is positioned upstream of the nucleic acid molecule encoding the precursor Complement Factor I protein or variant thereof and the nucleic acid molecule encoding the furin protein or variant thereof;
c. a translation initiation sequence, wherein the translation initiation sequence is positioned downstream of the promoter element and upstream of the nucleic acid molecule encoding the precursor Complement Factor I protein or variant thereof and the nucleic acid molecule encoding the furin protein or variant thereof.

In certain embodiments, the method comprises expressing the nucleic acid molecule encoding a recombinant precursor Complement Factor I protein or variant thereof and the nucleic acid molecule encoding a recombinant furin protein in a eukaryotic cell, wherein optionally the eukaryotic cell is a Human Embryonic Kidney 293T cell.

In certain embodiments, the method comprises expressing the recombinant precursor Complement Factor I protein and a recombinant furin protein in an expression system as described herein.

In certain embodiments, the method further comprises recovering the recombinant mature Complement Factor I protein.

In certain embodiments, the method is an in vitro method. In certain embodiments, the method is an in vivo method. Further details are provided herein below.

In a further aspect of the invention there is provided an expression vector as described herein for increasing the concentration of mature CFI within a human subject.

In a further aspect of the present invention there is provided an expression vector as described herein for use in the treatment and/or prevention of a Complement system mediated disorder.

Examples of a complement-mediated disease or disorder that may be treated by the present invention include, but are not limited to, atypical haemolytic uremic syndrome (aHUS); membranoproliferative glomerulonephritis Type 2 (MPGN2); microangiopathic hemolytic anaemia, Huntingdon's disease, C3 Glomerulopathy, cerebral inflammation, thrombocytopenia; Guillain-Barr6 syndrome, Multiple sclerosis (MS), Alzheimer's disease, Parkinson's disease, allergic encephalomyelitis, Myasthenia gravis (MG); systemic lupus erythematosus, systemic sclerosis, rheumatoid arthritis; cardiovascular diseases or conditions such as myocardial infarction, chronic cardiovascular disease, atherosclerosis or stroke; haematological disorders such as paroxysmal nocturnal haemoglobinuria; respiratory disorder such as asthma; dermatological diseases such as bullous pemphigoid or psoriasis; treatment following organ transplant rejection, graft versus host disease; ocular diseases or conditions such as age-related macular degeneration (AMD), glaucoma, diabetic retinopathy, early-onset macular degeneration, Stargardt's disease, central serous chorioretinopathy or retinitis pigmentosa, neuromyelitis optica or uveitis, or other inflammatory and/or autoimmune diseases.

Preferably, the complement system mediated disorder is an ocular disease or condition such as age-related macular degeneration (AMD), glaucoma, diabetic retinopathy, early-onset macular degeneration, Stargardt's disease, central serous chorioretinopathy or retinitis pigmentosa, neuromyelitis optica or uveitis, or other inflammatory and/or autoimmune diseases. Most preferably, the disorder is AMD. Further details of complement system mediated disorders are provided herein.

In a further aspect of the present invention there is provided an expression system as described herein for use in the treatment and/or prevention of a complement system mediated disorder. Examples of a complement-mediated disease or disorder that may be treated by the present invention include, but are not limited to, atypical haemolytic uremic syndrome (aHUS); membranoproliferative glomerulonephritis Type 2 (MPGN2); microangiopathic hemolytic anaemia, Huntingdon's disease, C3 Glomerulopathy, cerebral inflammation, thrombocytopenia; Guillain-Barr6 syndrome, Multiple sclerosis (MS), Alzheimer's disease, Parkinson's disease, allergic encephalomyelitis, Myasthenia gravis (MG); systemic lupus erythematosus, systemic sclerosis, rheumatoid arthritis; cardiovascular diseases or conditions such as myocardial infarction, chronic cardiovascular disease, atherosclerosis or stroke; haematological disorders such as paroxysmal nocturnal haemoglobinuria; respiratory disorder such as asthma; dermatological diseases such as bullous pemphigoid or psoriasis; treatment following organ transplant rejection, graft versus host disease; ocular diseases or conditions such as age-related macular degeneration (AMD), glaucoma, diabetic retinopathy, early-onset macular degeneration, Stargardt's disease, central serous chorioretinopathy or retinitis pigmentosa, neuromyelitis optica or uveitis, or other inflammatory and/or autoimmune diseases.

Preferably, the complement system mediated disorder is an ocular disease or condition such as age-related macular degeneration (AMD), glaucoma, diabetic retinopathy, early-onset macular degeneration, Stargardt's disease, central serous chorioretinopathy or retinitis pigmentosa, neuromyelitis optica or uveitis, or other inflammatory and/or autoimmune diseases. In preferred embodiments, the disorder is a complement mediated ocular disorder or disease. Most preferably, the disorder is AMD. Further details of complement system mediated disorders are provided herein.

In a further aspect of the present invention there is provided a method of treating and/or preventing a complement system mediated disorder in a subject, the method comprising administering the expression system as described herein to a subject.

In a further aspect of the present invention there is provided a method of treating and/or preventing a complement system mediated disorder in a subject, the method comprising administering the expression vector as described herein to a subject.

In certain embodiments, the method provides for production of mature Complement Factor I.

In certain embodiments, the method comprises a step of producing mature Complement Factor I from an expression vector.

In certain embodiments, the method provides for recombinant production of mature Complement Factor I from an expression vector as described herein in vivo.

In certain embodiments, the method provides for recombinant production of mature Complement Factor I in a subject.

In certain embodiments, the method produces a high concentration of recombinantly produced mature Complement Factor I.

In certain embodiments, the present invention provides a method for increasing the concentration of mature Complement Factor I within a cell.

In certain embodiments, the present invention provides a method for increasing the concentration of mature Complement Factor I within a tissue.

In certain embodiments, the present invention provides a Complement Factor I protein for use in the treatment of a complement system associated disorders.

In a further aspect of the present invention there is provided a mature recombinant Complement Factor I protein obtainable by a method described herein.

In a further aspect of the present invention there is provided a mixture of a mature recombinant Complement Factor I protein and a recombinant precursor complement Factor I protein obtainable by a method described herein, wherein the mixture comprises greater than 50% mature recombinant CFI protein: recombinant precursor CFI protein.

In certain embodiments, the mature recombinant Complement factor I protein is a mammalian Complement Factor I protein e.g. a human CFI protein.

In a further aspect of the present invention there is provided a therapeutic composition comprised of a mature recombinant Complement Factor I protein described herein and obtainable from a method described herein.

In certain embodiments, the composition is for use in the treatment of a subject suffering from a complement system mediated disorder e.g. atypical haemolytic uremic syndrome (aHUS); membranoproliferative glomerulonephritis Type 2 (MPGN2); microangiopathic hemolytic anaemia, Huntingdon's disease, C3 Glomerulopathy, cerebral inflammation, thrombocytopenia; Guillain-Barr6 syndrome, Multiple sclerosis (MS), Alzheimer's disease, Parkinson's disease, allergic encephalomyelitis, Myasthenia gravis (MG); systemic lupus erythematosus, systemic sclerosis, rheumatoid arthritis; cardiovascular diseases or conditions such as myocardial infarction, chronic cardiovascular disease, atherosclerosis or stroke; haematological disorders such as paroxysmal nocturnal haemoglobinuria; respiratory disorder such as asthma; dermatological diseases such as bullous pemphigoid or psoriasis; treatment following organ transplant rejection, graft versus host disease; ocular diseases or conditions such as age-related macular degeneration (AMD), glaucoma, diabetic retinopathy, early-onset macular degeneration, Stargardt's disease, central serous chorioretinopathy or retinitis pigmentosa, neuromyelitis optica or uveitis, or other inflammatory and/or autoimmune diseases.

Preferably, the complement system mediated disorder is an ocular disease or condition such as age-related macular degeneration (AMD), glaucoma, diabetic retinopathy, early-onset macular degeneration, Stargardt's disease, central serous chorioretinopathy or retinitis pigmentosa, neuromyelitis optica or uveitis, or other inflammatory and/or autoimmune diseases. Most preferably, the disorder is AMD.

In a further aspect of the present invention there is provided a method for separating a recombinant mature Complement Factor I (CFI) protein from one or more cellular components wherein the method comprises the following steps;
(a) contacting a preparation comprising a mixture of a precursor complement factor I protein, a mature form complement factor I protein and one or more further cell components with a chromatographic material under conditions that enable said precursor complement factor I protein and said mature form complement factor I protein to each bind to the chromatographic material;
(b) contacting the chromatographic material with one or more salt containing elution buffer solutions; and
(c) eluting said precursor complement factor I protein and mature complement factor I protein to obtain a series of eluates,

wherein, within the series of eluates, the precursor complement system protein and mature form complement system protein are substantially separated from one another, and/or wherein the precursor complement factor I protein and mature form complement factor I protein are substantially separated from other cellular components.

In certain embodiments, the chromatography material is an affinity chromatography material. In certain embodiments, the chromatography material comprises a chromatography material coupled to an OX21 monoclonal antibody.

In certain embodiments, the method further comprises the following steps;
d) contacting a preparation comprising the eluates comprising mature Complement Factor I protein and precursor Complement Factor I protein with at least one further chromatographic material under conditions that said precursor Complement Factor I protein and said mature form Complement Factor I protein bind to the at least one further chromatographic material;
e) contacting the at least one further chromatographic material with one or more salt containing elution buffer solutions; and
(f) eluting said precursor complement system protein and mature complement system protein; in order to obtain a further series of distinct eluates,

wherein, within the further series of eluates, the precursor complement factor I protein and mature form complement factor I protein are substantially separated from one another.

In certain embodiments, the further chromatography material is a cation-exchange (CEX) chromatography material.

In certain embodiments, the precursor complement factor I protein and mature form complement factor I protein are eluted from the cation-exchange chromatography by contacting the chromatography material onto which the precursor complement factor I protein and mature form complement factor I protein are bound by contacting the chromatography material with buffer solutions containing an increasing concentration of salt. In certain embodiment, the elution buffer solutions contain an increasing concentration of sodium chloride.

In certain embodiments, the salt concentration of the elution buffer solutions contacting the chromatography material onto which the precursor complement factor I protein and mature form complement factor I protein are bound is increased by a linear gradient. In certain embodiments, the salt concentration of the elution buffers is increased by a step gradient. In certain embodiments, the salt concentration of the elution buffers is increased by a linear gradient and/or a step gradient.

In certain embodiments, the elution buffer solutions contacting the cation exchange chromatography material have a pH of about 4.5-7.5, preferably optionally about pH 6.0.

In certain embodiments, the elution buffer solutions contacting the affinity chromatography material have a pH of about 1.5-4.5, optionally about pH 2.7.

In certain embodiments, the elution buffer solutions contacting the affinity chromatography material comprise glycine, wherein optionally the glycine is at a concentration of 0.1M.

In certain embodiments, the method comprises performing affinity chromatography (e.g. OX21 monoclonal antibody—NHS-Sepharose), anion-exchange (AEX) chromatography, and/or cation-exchange (CEX) chromatography to obtain the eluate.

In certain embodiments, the method comprises contacting a chromatographic material with an elution buffer solution with a salt concentration increased through a linear gradient. In certain embodiments, the elution buffer solution have at a pH of about 4.5-7.5, preferably about pH 6.0.

In certain embodiments, the buffer comprises a sodium or potassium salt. In certain embodiments, the precursor Complement Factor I and mature Complement Factor I are present in the preparation at a molar ratio of about 2.5:7.5.

### DETAILED DESCRIPTION OF CERTAIN EMBODIMENTS

### Brief Description of the Drawings

Certain embodiments of the present invention will now be described hereinafter, by way of example only, with reference to the accompanying drawings in which:

FIG. 1 depicts an overview of certain aspects of the complement system;

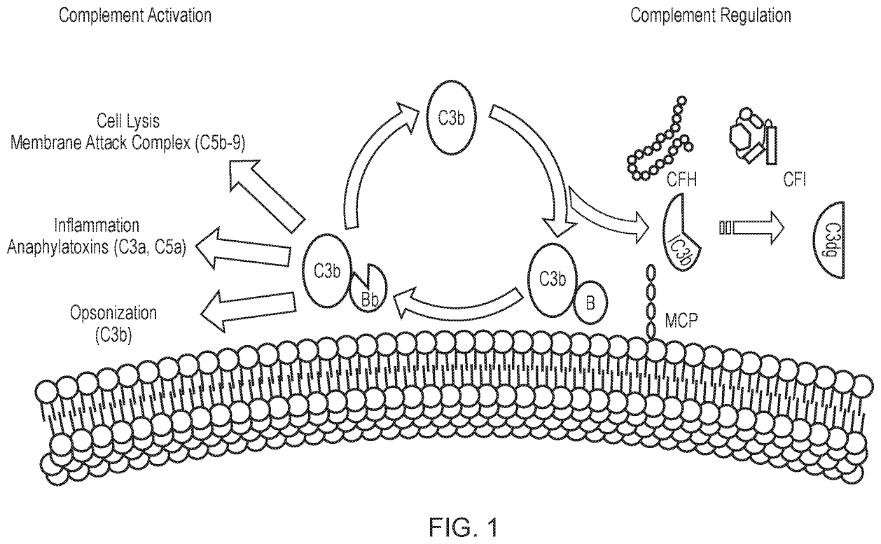

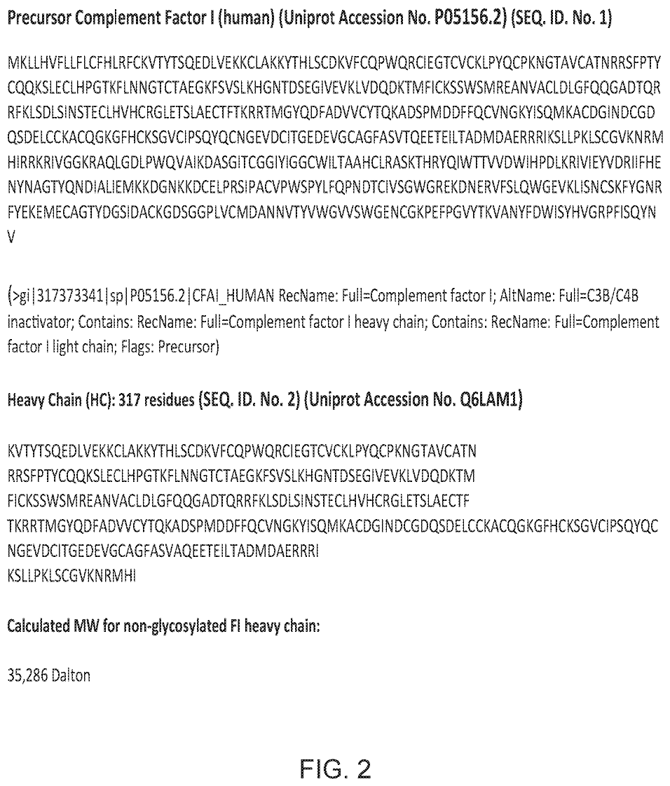

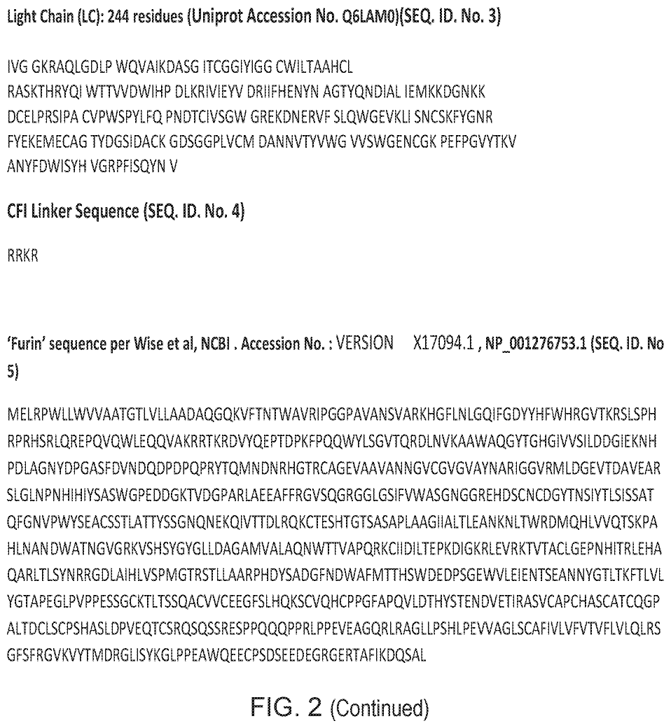

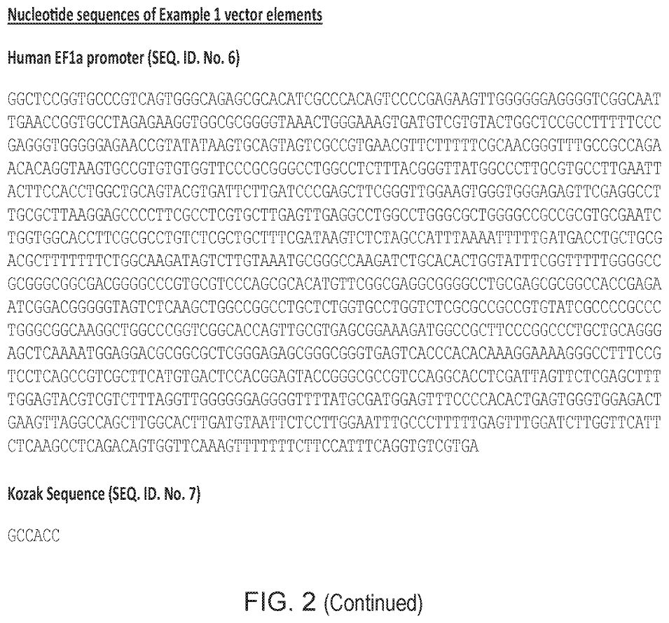

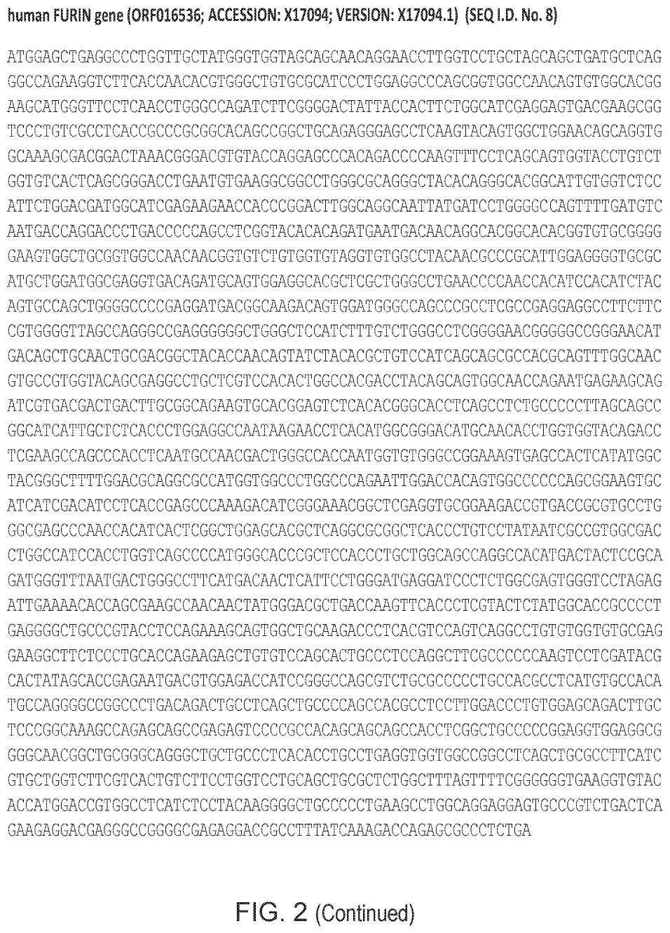

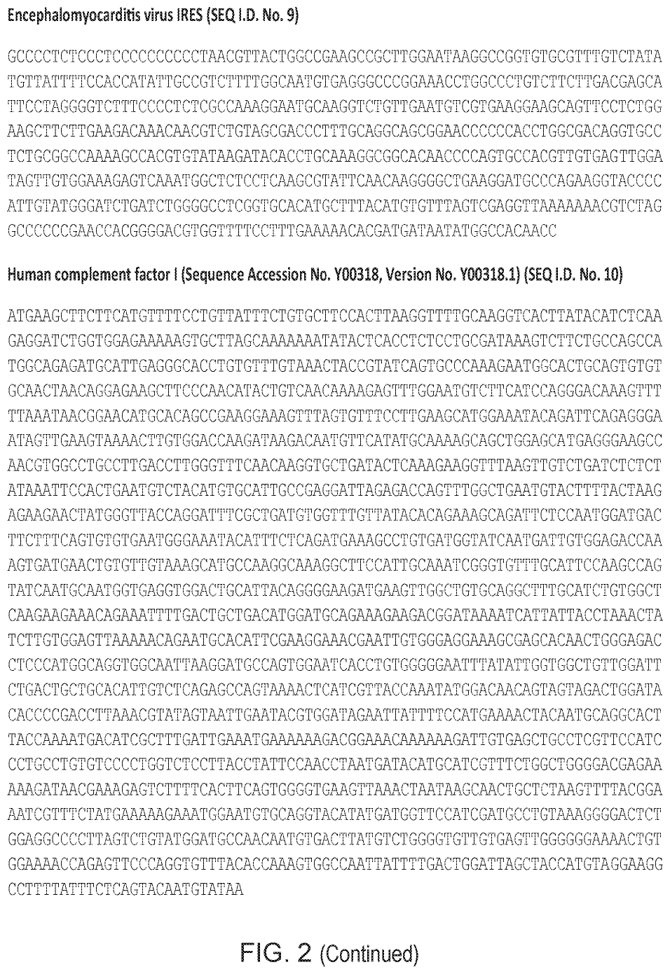

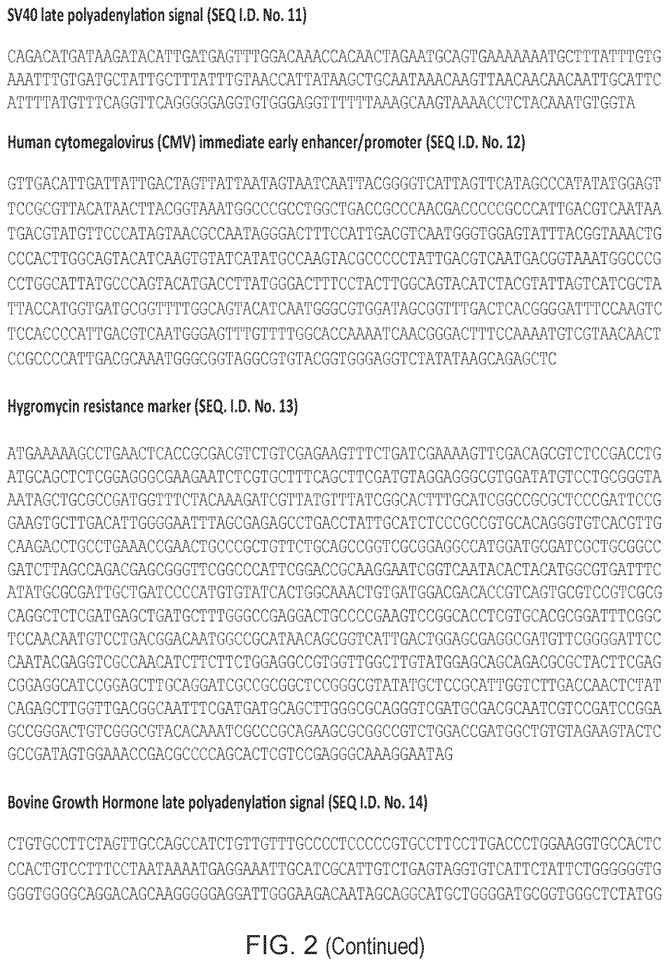

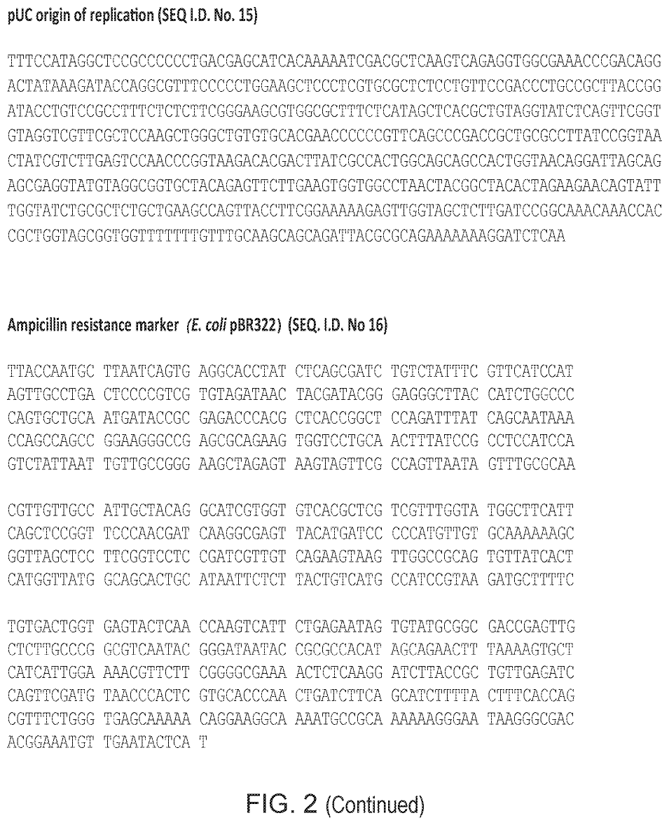

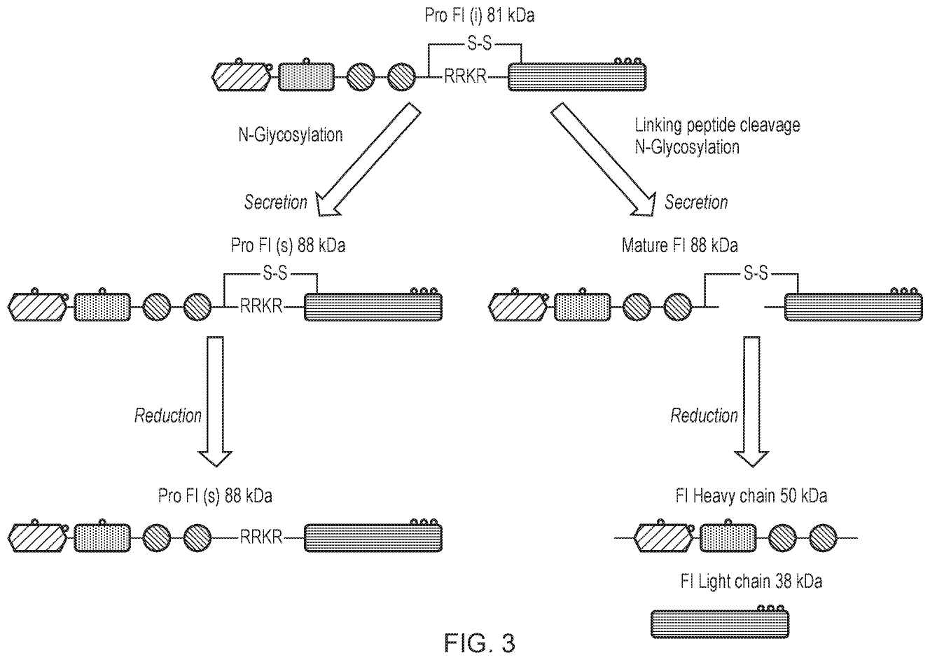

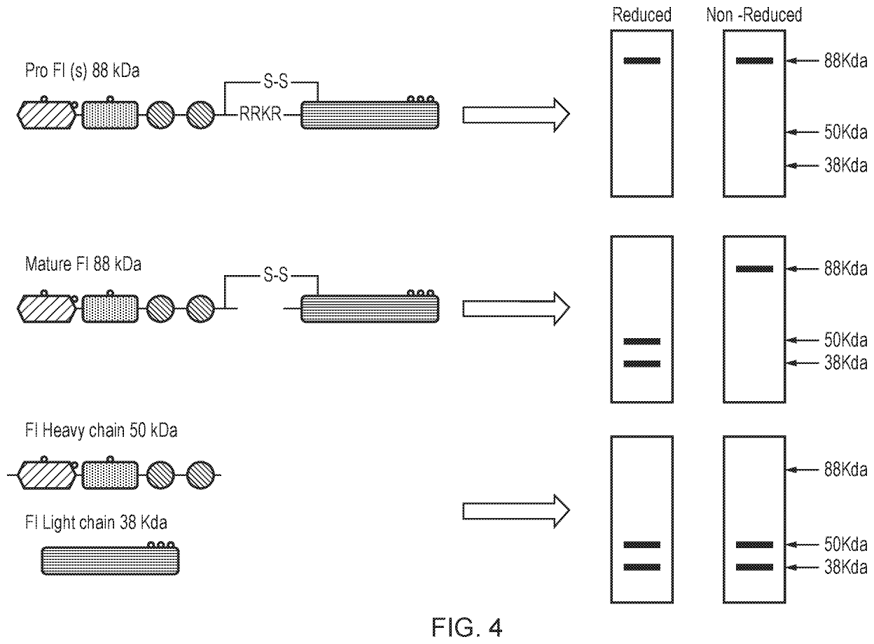

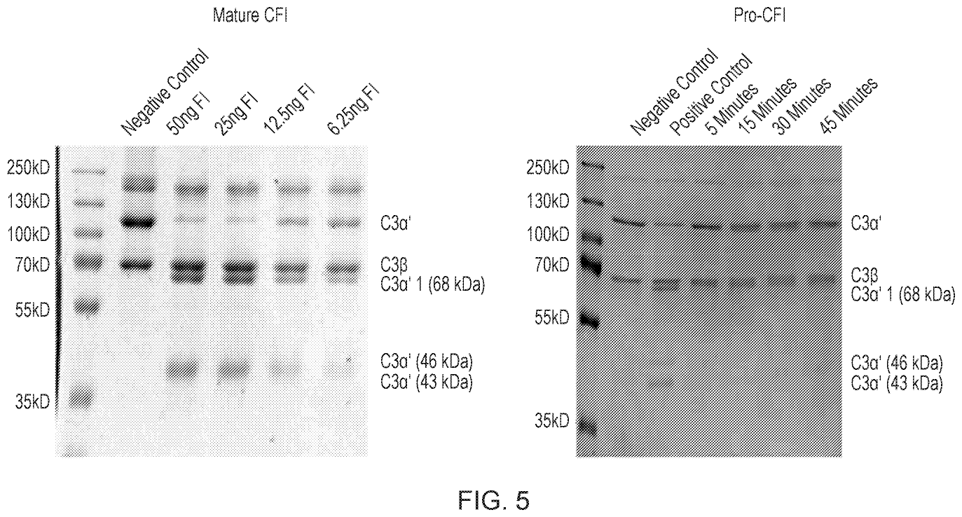

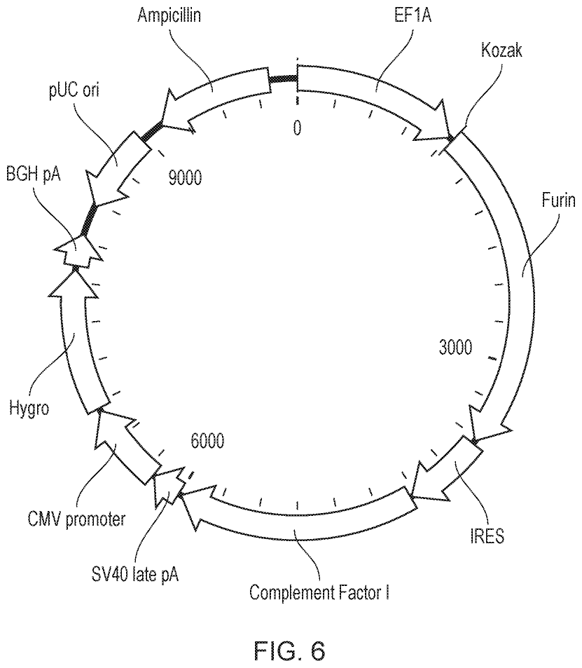

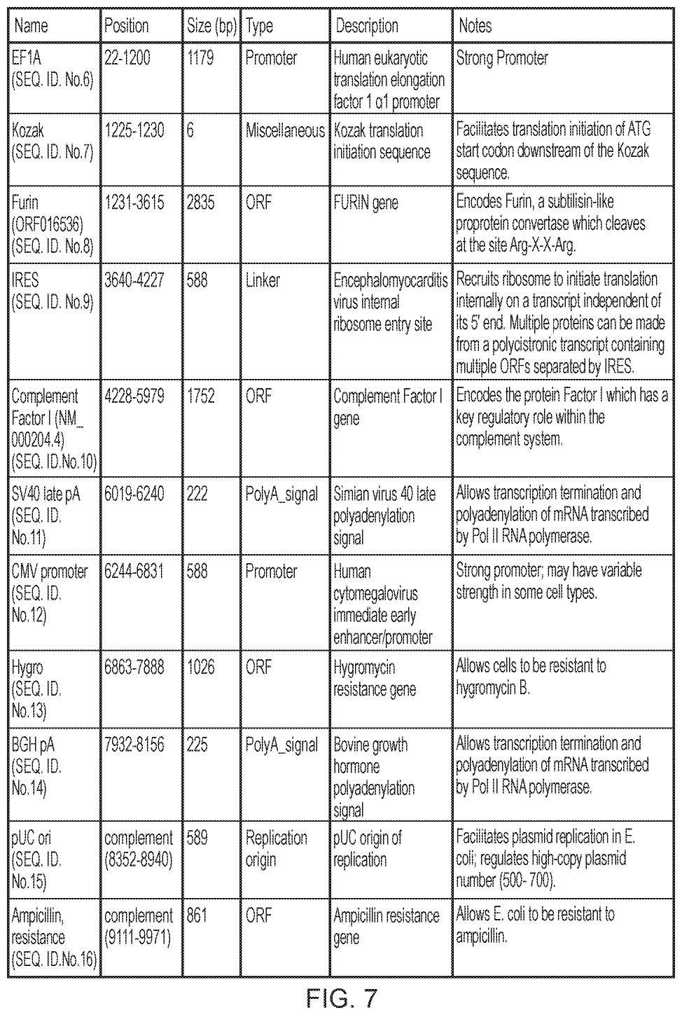

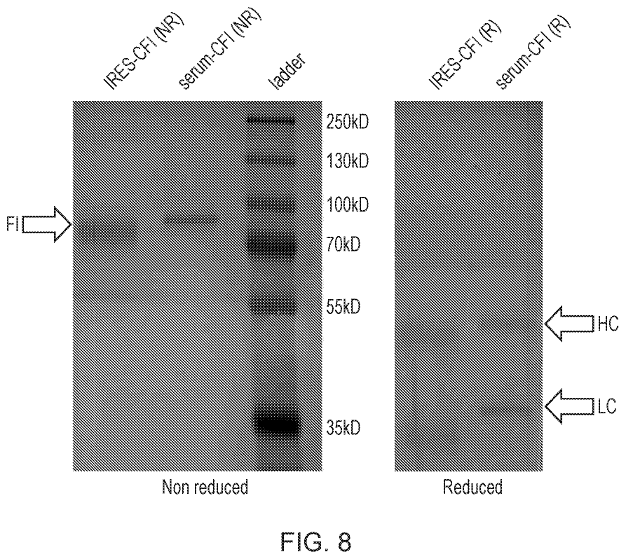

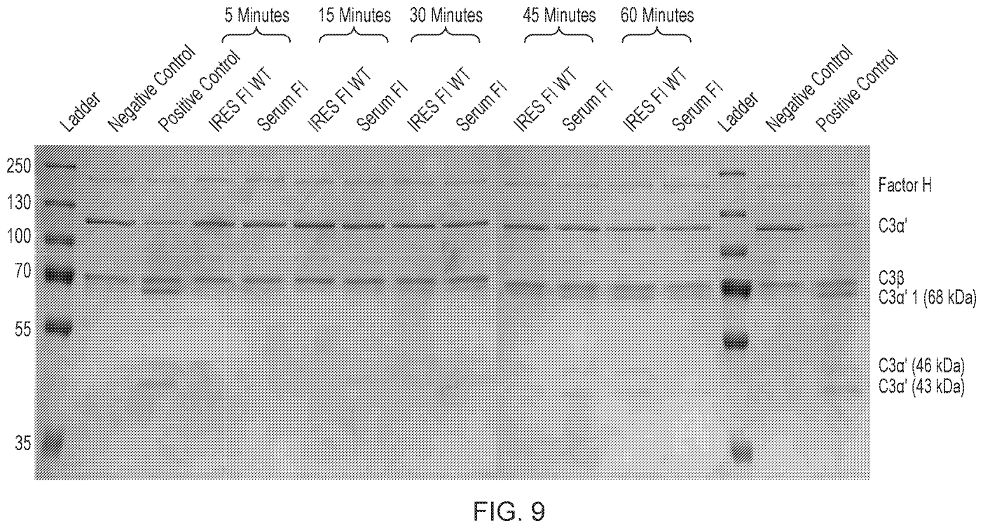

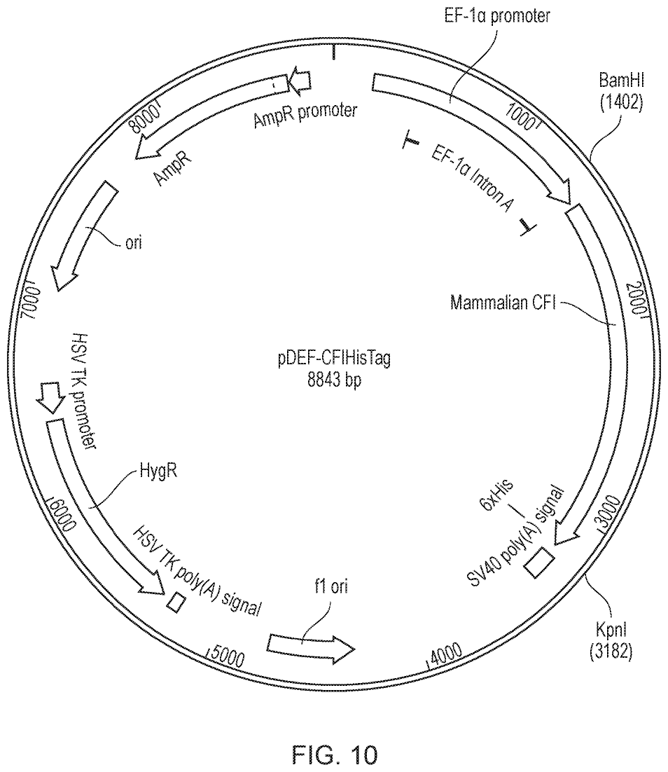

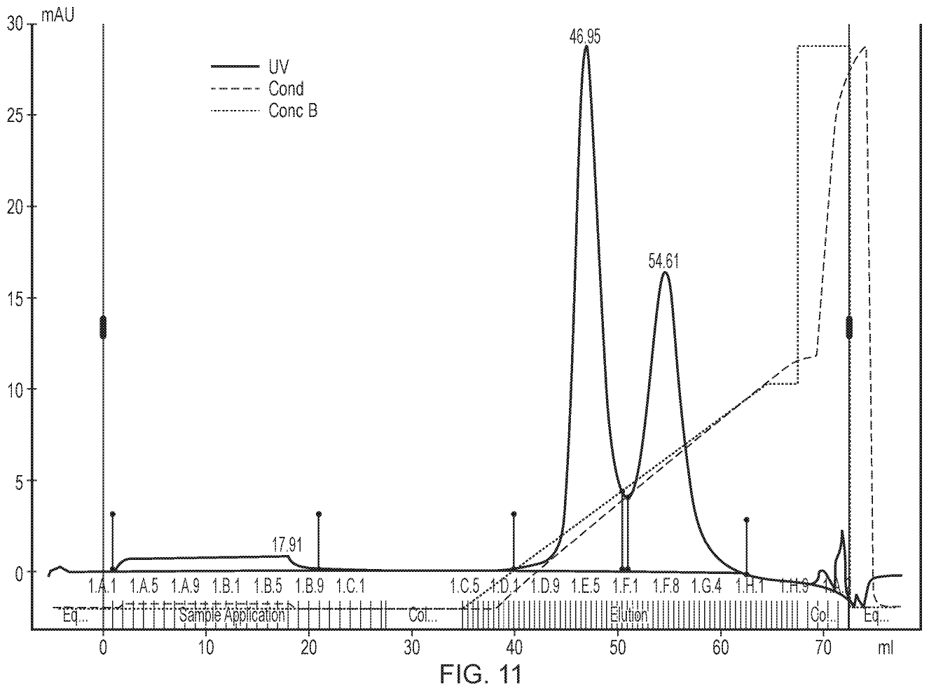

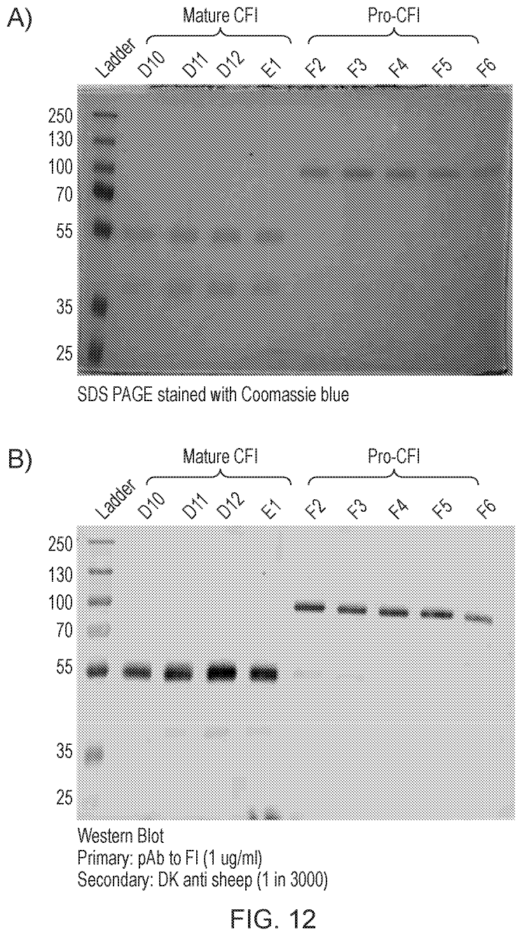

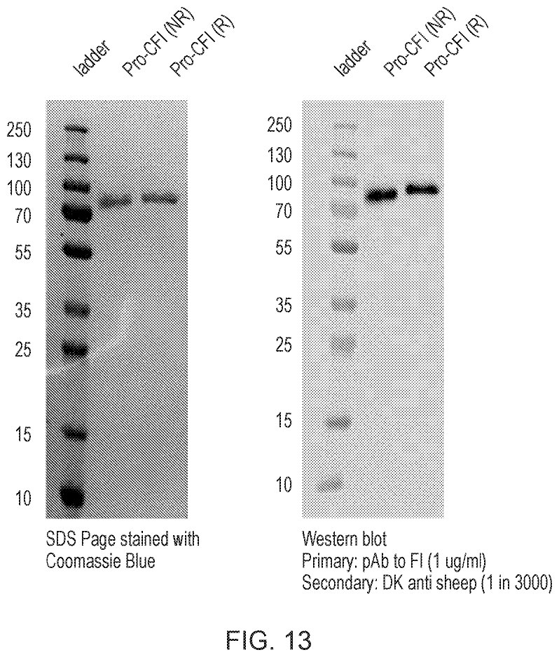

FIG. 2 depicts the amino acid sequences of proteins, and the nucleotide sequences of nucleic acid molecules described herein. Particularly:
SEQ. ID. No. 1 is an amino acid sequence of human precursor Complement factor I;
SEQ. ID. No. 2 is an amino acid sequence of human heavy chain of a mature complement Factor I;
SEQ. ID. No. 3 is an amino acid sequence of human light chain of a mature complement Factor I;
SEQ. ID. No. 5 is an amino acid sequence of a human furin protein; and
SEQ. ID. No. 4 is an amino acid sequence of a linker sequence of human complement factor I;
SEQ. ID. No. 6 is the nucleotide sequence of the Human EF1a promoter;
SEQ. ID. No. 7 is the nucleotide sequence of a kozak sequence;
SEQ. ID. No. 8 is the nucleotide sequence of human FURIN gene (ORF016536);
SEQ. ID. No. 9 is the nucleotide sequence of Encephalomyocarditis virus IRES;
SEQ. ID. No. 10 is the nucleotide sequence of Human complement factor I (Sequence Accession No. NM_000204, Version No. NM_000204.4);
SEQ. ID. No. 11 is the nucleotide sequence of SV40 late polyadenylation signal;
SEQ. ID. No. 12 is the nucleotide sequence of Human cytomegalovirus (CMV) immediate early enhancer/promoter;
SEQ. ID. No. 13 is the nucleotide sequence of the Hygromycin resistance marker;
SEQ. ID. No. 14 is the nucleotide sequence of the Bovine Growth Hormone late polyadenylation signal;
SEQ. No. 15 is the nucleotide sequence of the pUC origin of replication;
SEQ. No. 16 is the nucleotide sequence of the ampicillin resistance marker.
SEQ. No. 17 is the nucleotide sequence of an example CMV promoter.
SEQ. No. 18 is the nucleotide sequence of an example CAG promoter.
SEQ. No. 19 is an example WPRE nucleotide sequence.
SEQ. No. 20 is an example WPRE3 nucleotide sequence.
SEQ. No. 21 is the nucleotide sequence of an example Bovine Growth Hormone poly-A signal.
SEQ. No. 22 is the nucleotide sequence of a further example Bovine Growth Hormone poly-A signal.

FIG. 3 is a representation of the processing of recombinant human CFI (FI) in mammalian cell lines. Pro-CFI (“Pro FI”) undergoes processing before secretion. When CFI is expressed in cells incomplete processing of the protein results in the secretion of both Pro-CFI with an intact RRKR linker, and the mature CFI (“mature FI”) in which the heavy and the light chain is linked only by a disulphide bond.

FIG. 4 illustrates a schematic showing migration of CFI (“FI”) on an SDS-PAGE gel. Different forms of CFI appear on both non-reducing and reducing gels. Pro-CFI appears at 88 kDa under reducing and non-reducing conditions. Mature CFI appears at 88 kDa under non-reducing conditions, but when reduced will appear at 50 kDa and 38 kDa due to breakage of the disulphide bond.

FIG. 5 shows evaluation of fluid phase cofactor activity of precursor CFI (“Pro-factor I”) and mature CFI (“fully processed CFI”) C3b, FH and various concentrations of fully processed factor I were incubated at 37° C. for 30 minutes (left). At 30 minutes the lowest concentration of fully processed factor I (6.25ng) (right) had cleaved C3b as evidenced by decrease in C3α -110 band and increase in C3α-68,-46, and -43 bands. In contrast by 45 minutes 10ng of precursor CFI showed no demonstrable activity.

FIG. 6 shows a plasmid map of Furin-IRES-CFI vector according to certain embodiments of the invention. The Furin gene is responsible for encoding the Furin enzyme which cleaves RRKR linker of pro-CFI. The Furin gene is responsible for synthesis of the Furin enzyme which cleaves the RRKR linker found in pro-CFI. The internal ribosomal entry site (IRES) initiates translation in cap-independent manner, allowing synthesis of two proteins from a single polycistronic mRNA. CFI codes for the protein complement factor I. Further vector details are provided in FIG. 7.

FIG. 7 shows a table of the components present in the vector depicted in FIG. 6 according to certain embodiments of the present invention.

FIG. 8 shows a representative silver stain of SDS-PAGE comparing serum purified CFI (FI) against IRES-vector produced CFI under non-reducing and reducing conditions. A single band can be identified at 88 kDa for both IRES and serum CFI under non-reducing conditions. In contrast, under reducing conditions, two separate bands can be identified for both IRES and serum FI at 50 kDa and 38 kDa corresponding to the heavy chain (HC) and light chain, respectively.

FIG. 9 shows comparison of fluid phase cofactor activity between serum purified CFI (FI) and IRES-vector CFI. C3b, Factor H (FH) and 10ng of either FI or serum purified FI were incubated in solution at 37° C. for 1h. The reaction was stopped by the addition of reducing laemelli buffer at 5, 15, 30, 45, and 60-minute intervals. C3b breakdown was assessed by SDS-PAGE and Coomassie staining. A decrease of the C3α-110 band and the appearances C3α -68, -46, and -43 bands were indicative of inactivation of C3b through proteolytic cleavage. Activity was shown to be equivalent for both origins of CFI.

FIG. 10 shows a diagram of a pDEF-CFI vector, the pDEF-CFI construct is a modified pDR2 EF1a (pDEF) expression vector with an inserted mammalian CFI sequence.

FIG. 11 illustrates a representative UV trace obtained when recombinant pro-CFI was purified from a recombinant protein preparation as delineated in Example 3.

FIG. 12 shows a representative SDS-PAGE of selected fractions obtained as per FIG. 11, run under reducing conditions, resolving mature CFI and pro-CFI. A) Coomassie Blue stained SDS PAGE—two bands are present for the samples corresponding to mature CFI, at 50 KDa and 38 kDa for the heavy and light chain, respectively. Samples containing pro-CFI exhibited a single band seen at 90 kDa B) Western blot detected using the polyclonal antibody to complement factor I. For mature CFI two bands are seen for the heavy and lights chains as in (A). For Pro-CFI only one band is observed.

FIG. 13 shows a representative SDS PAGE resolving pro-CFI visualised with Coomassie and Western Blot. Under both reducing (R) and non-reducing (NR) conditions there is one band present.

Unless defined otherwise, all technical and scientific terms used herein have the same meaning as commonly understood by a person skilled in the art to which this invention belongs.

Certain aspects of the present invention provide for expression of a precursor complement system protein with co-expression of a protease. In certain embodiments, the co-expressed protease can interact with the precursor Complement System Protein to produce a mature form Complement System Protein.

Certain aspects of the present invention provide a method of providing a recombinant mature Complement Factor I (CFI) protein. Further aspects of the present invention provide an expression system for expressing a recombinant mature Complement Factor I protein.

Certain aspects of the present invention relate to a vector which provides expression of recombinant precursor Complement Factor I and furin from polycistronic RNA such that a mature form Complement Factor I is produced.

Certain aspects of the present invention provide an isolated recombinant mature Complement system protein. Certain aspects of the present invention provide an isolated recombinant mature Complement Factor I protein.

The term ‘Complement system protein’ refers to any protein involved in the complement cascade. The complement cascade and complement system proteins are described in the art.

In certain embodiments, the recombinant mature Complement system protein may be a Complement cascade regulatory protein.

Certain aspects of the present invention provide an isolated recombinant mature Complement Factor I.

In a further aspect of the present invention, there is provided an expression system for expressing a recombinant CFI protein and a serine protease protein. The expression system may be for use in vivo.

As used herein, an “expression system” refers to a system comprising components useful for the expression of recombinant proteins. For example, an expression system may comprise one or more expression vectors. In certain embodiments, an expression system may further comprise a biological environment, e.g. a cell, which can be used to provide energy and machinery for protein synthesis. It is also possible to use cellular extracts containing the necessary components, i.e., cell-free protein expression systems.

Additionally, the expression system may comprise a vector (also referred to herein as an expression vector) that facilitates the introduction of genetic material into the cell; containing regulatory parts that provide for replication of the genetic material and usually selection markers for maintenance. Also, the expression system may comprise a nucleic acid molecule which is incorporated into the vector that contains the open reading frame encoding the amino acid sequence for the protein(s) to be expressed. The vector may also contain the components necessary for transcription and translation of the protein to be produced. Further details of the vector are provided herein.

Appropriately, the expression system may comprise a host cell and/or tissue. The host cell may be an ex-vivo host cell or tissue. Alternatively, the expression system may be for in vivo use e.g. as a gene therapy. Thus, an expression system may be exogenously administered to a subject in need thereof.

As used herein, the subject is preferably a human subject.

In certain embodiments, the expression system is an in vitro expression system and/or an ex vivo expression system.

In certain embodiments, the expression system is an in vivo expression system.

The term “isolated” as used herein refers to a biological component (such as a nucleic acid molecule or protein) that has been substantially separated or purified away from other biological components in the cell of the organism in which the component naturally occurs, i.e., other chromosomal and extra chromosomal DNA and RNA, and proteins. Nucleic acids and proteins that have been “isolated” include nucleic acids and proteins purified by standard purification methods. The term also embraces nucleic acids and proteins prepared by recombinant expression in a host cell as well as chemically synthesized nucleic acids, proteins, and peptides.

As used herein, the term “protein” can be used interchangeably with “peptide” or “polypeptide”. Aptly the term “protein” means at least two covalently attached alpha amino acid residues linked by a peptidyl bond. The term protein encompasses purified natural products, or chemical products, which may be produced partially or wholly using recombinant or synthetic techniques. The term protein may refer to a complex of more than one polypeptide, such as a dimer or other multimer, a fusion protein, a protein variant, or derivative thereof. The term also includes modified proteins, for example, a protein modified by glycosylation, acetylation, phosphorylation, pegylation, ubiquitination, and so forth. A protein may comprise amino acids not encoded by a nucleic acid codon.

As used herein, the term ‘precursor’ refers to a polypeptide subject to further posttranslational processing. In certain instances, a precursor polypeptide is less active relative to its corresponding mature form. Also used herein is the term “proform”, and the term “proform” and “precursor” are used interchangeably herein.

A precursor form of the protein may be processed to form a mature form of protein by a protease. As used herein, the term ‘protease’ refers to any protein capable of hydrolysing a peptide bond. The term ‘endoprotease’ refers to a protease capable of hydrolysing non-terminal amino acid peptide bonds. The term ‘serine endoprotease’ refers to an endoprotease wherein a nucleophilic serine serves as the enzymes active centre. In certain embodiments, the protease is a furin protein. The protease may be a mammalian protease e.g. a human protease. The protease may be human furin.

The term ‘mature’ as used herein, refers to a polypeptide or protein that is derived from a precursor polypeptide that has undergone post translational modifications to a species with differing activity relative to the precursor polypeptide.

As used herein, the terms “transgene expression cassette” and “expression cassette” is used to refer to a nucleic acid molecule which comprise gene sequences that a nucleic acid vector is to deliver to target cells. These sequences may include the gene of interest (e.g., CFI, furin genes), one or more promoters, and regulatory elements.

As used herein, the term “regulatory elements” may refer to regulatory elements that are necessary for effective expression of a gene in a target cell (e.g., CFI, furin genes), and thus should be included in a transgene expression cassette. Such sequences could include, for example, promoters, polyadenylation sequences, enhancer sequences, polylinker sequences facilitating the insertion of a DNA fragment within a plasmid vector, or sequences responsible for intron splicing and polyadenlyation of mRNA transcripts, etc.

It is considered that the present inventors have devised a method of recombinantly producing a mature Complement system protein, for example a recombinant mature CFI protein within a host cell.

In some embodiments, the invention provides for a method of recombinantly producing a mature Complement system protein, for example a recombinant mature CFI protein which is substantially isolated from other cellular components.

It is considered that prior art methods of producing a recombinant CFI protein have resulted in incomplete processing of a precursor CFI protein such that a recombinant mature CFI protein has not been substantially isolated from precursor CFI, or such that recombinant precursor CFI requires in vitro incubation with furin. It is considered that the present inventors have devised a method of recombinantly producing mature CFI protein with a reduced number of processing steps compared to methods disclosed in the prior art.

Wong et al showed that co-transfection of COS-1 cells with two distinct vectors independently encoding human pro-CFI and furin only resulted in around synthesis of 50% mature CFI. Furthermore, the independent pro-CFI and furin vector design generally precludes the use of such an expression system in gene therapy due to the requirement, and unlikely event, of co-transfection of cells within perfuse tissue.

Thus, certain embodiments of the present invention relate to production and isolation of a mature complement system protein.

Certain embodiments of the present invention relate to production and isolation of recombinant mature complement factor I (CFI).

In certain embodiments, the method comprises recombinant co-expression of a precursor form of a complement system protein with a protease enzyme that is capable of processing the precursor form of a complement system protein to form the mature form of the complement system protein.

In certain embodiments, the method comprises co-expression of a precursor of a complement system protein and a protease enzyme from the same expression vector. In certain embodiments, the method comprises co-expression of a precursor of a complement system protein and a protease enzyme from polycistronic RNA.

In certain embodiments, the method comprises recombinant co-expression of precursor CFI protein and a protease enzyme capable of processing pro-CFI to produce mature CFI.

In certain embodiments, the method comprises co-expression of precursor CFI protein and a protease enzyme from the same expression vector. In certain embodiments, the method comprises co-expression of precursor CFI protein and protease enzyme from polycistronic RNA. In certain embodiments, the protease enzyme is an endoprotease. In certain embodiments, the endoprotease is a serine endoprotease. In certain embodiments, the serine endoprotease is furin.

Complement Factor I is an important complement regulator. It is expressed in numerous tissues but principally by liver hepatocytes. CFI is a heterodimer in which the two chains are linked together by disulphide bond. The heavy chain contains the Factor I module, a CD5 domain and two low density lipoprotein receptor domains (LDLr). The light chain comprises a serine protease domain, the active site of which consists of a triad of His380, Asp439 and Ser525. A CFI heavy chain amino acid sequence is shown in SEQ ID. No. 2 and a CFI light chain amino acid sequence is shown in SEQ ID. No 3.

When CFI is synthesised, it is initially made as a single chain precursor (precursor CFI protein), in which a four-residue linker peptide (RRKR) connects the heavy chain to the light chain. Thus, as used herein, the term “precursor CFI protein” is used to refer to a single chain precursor complement Factor I protein which comprises a four-residue linker peptide (RRKR).

Aptly, the precursor CFI protein (pro-CFI) is substantially inactive and has essentially no C3, C3b-inactivating or iC3b-degradation activity. In certain embodiments, the recombinant precursor CFI protein comprises an amino acid sequence as set forth in SEQ. ID. No. 1.

During processing, the precursor CFI protein is cleaved by a calcium-dependent serine endoprotease, furin, leaving the heavy chain and light chain of full-length mature CFI held together by a single disulphide bond. This protein is referred to herein as a mature CFI protein. In certain embodiments, furin comprises an amino acid sequence as set forth in SEQ. ID. No. 5.

Thus, as used herein, the term “mature CFI protein” refers to a CFI protein which is or has been cleaved at or adjacent to a RRKR linker sequence e.g. by furin. In certain embodiments, the mature CFI protein lacks an RRKR linker sequence as compared to a precursor CFI protein, wherein the precursor CFI protein comprises a RRKR linker sequence at positions 318 to 321. In other embodiments, the mature CFI protein is cleaved adjacent to the RRKR linker sequence and therefore the mature CFI protein may comprise a light chain and a heavy chain, one or both of which comprises one or more amino acid residues of the linker sequence.

In certain embodiments, the recombinant precursor CFI protein is a non-human mammalian CFI protein. In other embodiments, the recombinant precursor CFI protein is a human CFI protein.

In certain embodiments, a mature CFI protein comprises a disulphide bond and wherein the recombinant mature CFI protein is cleavable into a heavy chain and a light chain upon reduction of the disulphide bond. In certain embodiments, the mature CFI protein comprises a heavy chain comprising a Factor I membrane attack complex (FIMAC) module, a CD5 module, and two LDLr modules, and a light chain comprising a serine protease domain. In certain embodiments, the mature CFI protein is glycosylated.

As used herein, the term “recombinant precursor CFI protein” is used to refer to a precursor CFI protein as described above which is obtained using recombinant methods.

As used herein, the term “recombinant mature CFI protein” is used to refer to a mature CFI protein as described above which is obtained using recombinant methods.

As used herein, the term “total CFI protein content” refers to a total content of the combination of recombinant mature CFI protein and a recombinant precursor CFI protein present in a single composition or preparation.

Aptly, a “recombinant mature CFI protein” is a mature CFI protein defined above which is made by recombinant expression, i.e. it is not naturally occurring or derived from plasma. Aptly, a wild-type mature CFI protein comprises two chains, each chain undergoing glycosylation which results in a total of six N-linked glycosylation sites which adds approximately 18 kDa of carbohydrate to the predicted molecular weight of 66 kDa.

The recombinant mature CFI protein may have a different glycosylation pattern to a naturally derived i.e. plasma-derived mature CFI protein.

In certain embodiments, the recombinant CFI protein is a fragment of a full length CFI protein which retains C3b-inactivating and iC3b-degradation activity.

The terms “recombinant” and “recombinant expression” are well-known in the art. The term “recombinant expression”, as used herein, relates to transcription and translation of an exogenous gene in a host organism.

Exogenous DNA refers to any deoxyribonucleic acid that originates outside of the host cell. The exogenous DNA may be integrated in the genome of the host or expressed from a non-integrating element.

A recombinant protein includes any polypeptide expressed or capable of being expressed from a recombinant nucleic acid. Thus, a recombinant precursor CFI protein is expressed by a recombinant DNA sequence. Recombinant precursor CFI protein may undergo enzymatic processing during expression at or adjacent to a RRKR linker sequence to leave a heterodimer as described herein.

In certain embodiments, the protease may be recombinantly co-expressed with recombinant complement system protein.

In certain embodiments, the protease may be recombinantly co-expressed with recombinant pro-CFI. In certain embodiments, the protease is a Furin protein.

Furin is a subtilisin-like proprotein convertase which cleaves protein in vivo at a minimal cleavage site of Arg-X-X-Arg. A human furin protein comprises an amino acid sequence as set forth in SEQ. ID. 5.

In certain embodiments, the Furin protein is a human furin protein or fragment thereof. In certain embodiments, the furin protein is a fragment of a mature Furin protein. Aptly, the Furin protein is a truncated Furin protein which is terminated before the transmembrane domain. Aptly the truncated Furin protein comprises at least one or more amino acid residues at a position at or between 595-791 that is involved in the catalytic activity of Furin e.g. to cleave at a RRKR linker sequence.

In certain embodiments, the Furin protein or fragment thereof is glycosylated. Aptly, the Furin protein or fragment thereof is glycosylated at one or more amino acid residues selected from Asn387, Asn440 and Asn553.

In certain embodiments, the Furin protein or fragment thereof has a molecular weight of 60 kDa or greater. Aptly, the Furin protein or fragment thereof has a molecular weight of between about 65 to 85 kDa. In certain embodiments, the Furin protein or fragment thereof comprises a tag e.g. a His tag.

In certain embodiments, the Furin protein or fragment thereof comprises the amino acid sequence as set forth in SEQ. ID. No. 5 or a fragment thereof. In certain embodiments, the furin protein fragment comprises at least amino acid residues 108 to 715 of a protein comprising the amino acid sequence as set forth in SEQ. ID. No: 5.

In certain embodiments, the Furin protein is a protein having at least 70% or 80%, e.g. at least 70%, 75%, 80%, 85%, 90%, 91%, 92%, 93%, 94%, 95%, 96%, 97%, 98%, 99% or 100% sequence identity with a protein having a sequence as depicted in SEQ. ID. No. 5. Aptly, the % sequence identity is over the entire length of the amino acid sequence set forth in SEQ. ID. No. 5. In certain embodiments, the Furin protein is a protein having at least 70%, 80%, 85%, 90%, 91%, 92%, 93%, 94%, 95%, 96%, 97%, 98%, 99% or 100% sequence identity with the sequence consisting of amino acid residues 108 to 715 of SEQ. ID. No. 5.

In certain embodiments, both recombinant pro-CFI and recombinant Furin are co-expressed from the same expression vector. In certain embodiments, recombinant pro-CFI and recombinant Furin are expressed from polycistronic RNA.

In certain embodiments, the expression vector may contain internal ribosome entry sequence (IRES). In certain embodiments, an IRES will be positioned between the genes encoding pro-CFI and a protease enzyme.

In certain embodiments, the gene encoding pro-CFI is upstream of the gene encoding furin protein. In preferred embodiments, the gene encoding pro-CFI is downstream of the gene encoding furin protein.

In certain embodiments, the gene encoding pro-CFI is upstream of the gene encoding furin protein, wherein an IRES is positioned between the genes encoding pro-CFI and furin protein.

In preferred embodiments, the gene encoding pro-CFI is downstream of the gene encoding furin protein, wherein an IRES is positioned between the genes encoding pro-CFI and furin protein.

In certain embodiments, the vector contains a promoter element upstream of the gene encoding a complement system protein. In other embodiments, the expression vector may contain promoters upstream of one or more protein encoding genes.

In certain embodiments, the expression vector contains a promoter element upstream of the gene encoding pro-CFI. In other embodiments, the expression vector may contain promoters upstream of one or more protein encoding genes.

In certain embodiments, the recombinant mature CFI protein comprises two polypeptides having respective amino acid sequences as set forth in SEQ. ID. No. 2 and SEQ. ID. No. 3, wherein the two amino acid sequences are linked by a disulphide bond.

In certain embodiments, the recombinant mature CFI protein comprises a first and a further polypeptide comprising respective amino acid sequences with at least 70%, 80% or 85% sequence identity to the respective amino acid sequences set forth in SEQ. ID. No. 2 and SEQ. ID. No. 3, wherein the first and further polypeptides are linked by a disulphide bond.

In certain embodiments, the recombinant mature CFI protein comprises a first and a further amino acid polypeptide which comprise a respective amino acid sequence having at least 90% sequence identity to the respective amino acid sequences set forth in SEQ ID NO: 2 and SEQ. ID. No 3, e.g. at least 91%, 92%, 93% or 94% identical, wherein the first and further amino acid sequences are linked by a disulphide bond.

In certain embodiments, the recombinant mature CFI protein comprises a first and a further polypeptide which comprise a respective amino acid sequence having at least 95% identity to the respective amino acid sequences set forth in SEQ ID NO: 2 and SEQ. ID. No. 3, e.g. at least 96%, 97%, 98% or 99% identity, wherein the first and further amino acid sequences are linked by a disulphide bond.

In certain embodiments, the recombinant mature CFI protein comprises a fragment of the amino acid sequence as set forth in SEQ. ID. No. 2 in which retains C3b-inactivating and iC3b-degradation activity.

In certain embodiments, the recombinant mature CFI protein comprises a fragment of the amino acid sequence as set forth in SEQ. ID. No. 3 in which retains C3b-inactivating and iC3b-degradation activity.

In certain embodiments, proteins having minor modifications in the sequence may be equally useful, provided they are functional. The terms “sequence identity”, “percent identity” and “sequence percent identity” in the context of two or more nucleic acids or polypeptides, refer to two or more sequences or sub-sequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity can be measured using sequence comparison software or algorithms or by visual inspection.

Various algorithms and software are known in the art that can be used to obtain alignments of amino acid or nucleotide sequences. Suitable programs to determine percent sequence identity include for example the BLAST suite of programs available from the U.S. government's National Center for Biotechnology. Information BLAST web site (http://blast.ncbi.nlm.nih.gov/Blast.cgi). Comparisons between two sequences can be carried using either the BLASTN or BLASTP algorithm. BLASTN is used to compare nucleic acid sequences, while BLASTP is used to compare amino acid sequences. ALIGN, ALIGN-2 (Genentech, South San Francisco, Calif.) or MegAlign, available from DNASTAR, are additional publicly available software programs that can be used to align sequences. One skilled in the art can determine appropriate parameters for maximal alignment by particular alignment software. In certain embodiments, the default parameters of the alignment software are used.

In certain embodiments, the recombinant mature Complement Factor I protein may comprise an amino acid sequence comprising one or more mutations as compared to a reference sequence. In certain embodiments, the reference sequence is as shown in SEQ. ID. No. 2 and 3. In certain embodiments, the reference sequence is as shown in SEQ. ID. No. 1. In certain embodiments, the mutation may be an insertion, a deletion, or a substitution.

Substitutional variants of proteins include those in which at least one amino acid residue in the amino acid sequence has been removed and a different amino acid residue inserted in its place. The mature recombinant CFI protein of certain embodiments of the present invention can contain conservative or non-conservative substitutions. The term “conservative substitution” as used herein relates to the substitution of one or more amino acid residues for amino acid residues having similar biochemical properties. Typically, conservative substitutions have little or no impact on the activity of a resulting protein. Screening of variants of the CFI proteins described herein can be used to identify which amino acid residues can tolerate an amino acid residue substitution. In one example, the relevant biological activity of a modified protein is not decreased by more than 25%, preferably not more than 20%, especially not more than 10%, compared with CFI when one or more conservative amino acid residue substitutions are affected.

In certain embodiments, the recombinant mature CFI proteins described herein are produced and characterised in one or more functional assays to determine the impact of CFI mutations on CFI activity. The recombinant mature CFI protein produced and/or characterised may, for example, be a G119R, L131R, V152M, G162D, R187Y, R187T, T2031, A240G, A258T, G287R, A300T, R317W, R339Q, V412M, or P553S variant, or any other variant wherein loss of function is suspected or impact on function is to be determined.

The functional activity of Complement Factor I, or a fragment, variant or derivative thereof, may be determined using any suitable method known to the skilled person. For example, measurement of Complement Factor I proteolytic activity is described in Hsiung et al. (Biochem. J. (1982) 203: 293-298). Both haemolytic and conglutinating assays for CFlactivity are described in Lachmann PJ & Hobart MJ (1978) “Complement Technology” in Handbook of Experimental Immunology 3rd edition Ed DM Weir Blackwells Scientific Publications Chapter 5A p17. A more detailed description, also including a proteolytic assay, is given by Harrison RA (1996) in “Weir's Handbook of Experimental Immunology” 5th Edition Eds; Herzenberg Leonore A'Weir DM, Herzenberg Leonard A & Blackwell C Blackwells Scientific Publications Chapter 75, 36-37. The conglutinating assay is highly sensitive and can be used for detecting both the first (double) clip converting fixed C3b to iC3b and acquiring reactivity with conglutinin; and for detecting the final clip to C3dg by starting with fixed iC3b and looking for the loss of reactivity with conglutinin. The haemolytic assay is used for the conversion of C3b to iC3b, and the proteolytic assay detects all the clips.

In certain embodiments, the purified mature complement system protein e.g. purified complement Factor I may be comprised in a composition that is essentially free of a protease protein or fragments thereof. In certain embodiments, the purified mature complement system protein may be comprised a pharmaceutical composition. In certain embodiments, said pharmaceutical composition further comprises one or more pharmaceutically acceptable excipients.

In certain embodiments, the complement system protein is a precursor CFI protein. In certain embodiments, the recombinant precursor CFI protein is a human precursor CFI protein. The recombinant precursor CFI protein comprises an amino acid sequence as set forth in SEQ. ID. No: 1.

In certain embodiments, the recombinant precursor complement system protein comprises a tag. In certain embodiments, the tag is a His-tag.

In certain embodiments, the recombinant precursor CFI protein is expressed in a eukaryotic cell. The protein may be expressed in vitro.

In certain embodiments, the method comprises expressing the recombinant precursor CFI protein in a prokaryotic cell. Aptly, the prokaryotic cell is Escherichia coli. In other embodiments, the prokaryotic cell may also be B. subtilis.

In certain embodiments, the eukaryotic cell is selected from an insect, a plant and a mammalian cell.

Suitable host cells for cloning or expressing the DNA encoding a CFI protein include prokaryote, yeast, or higher eukaryote cells.

Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis, Pseudomonas such as P. aeruginosa, and Actinobacteria such as Streptomyces species.

In addition to prokaryotes, eukaryotic microbes such as filamentous fungi or yeast may be suitable cloning or expression hosts for complement system protein encoding vectors, complement system protein and protease encoding vectors, CFI encoding vectors, or CFI and Furin encoding vectors.

Saccharomyces cerevisiae, or common baker's yeast, is the most commonly used among lower eukaryotic host microorganisms although others may be useful.

In certain embodiments, the host cell is a mammalian host cell e.g. monkey kidney CV1 line transformed by SV40 (e.g. COS-7); human embryonic kidney line (e.g. 293 or 293T cells); baby hamster kidney cells (e.g. BHK); Retinal pigment epithelium cells; Chinese hamster ovary cells/-DHFR (CHO), mouse sertoli cells (e.g. TM4); monkey kidney cells (e.g. CV1); African green monkey kidney cells (e.g. VERO-76); human cervical carcinoma cells (e.g. HELA); canine kidney cells (e.g. MDCK); buffalo rat liver cells (e.g. BRL 3A); human lung cells (e.g. W138); human liver cells (e.g. Hep G2); mouse mammary tumor (MMT 060562); TRI cells, MRC 5 cells and FS4 cells.

Host cells are transformed with the expression or cloning vectors described herein for protein production and cultured in conventional nutrient media modified as appropriate for inducing promoters, selecting transformants, or amplifying the genes encoding the desired sequences.

In certain embodiments, the expression vector and/or system is for in vivo expression. Thus, in certain embodiments, the expression system comprises a vector adapted for in vivo expression. In certain embodiments, the vector is a viral vector. In certain embodiments the vector is a non-viral vector.

The terms “nucleic acid molecule‘ or’ nucleic acid sequence”, as used herein, refers to a polymer of nucleotides in which the 3′ position of one nucleotide sugar is linked to the 5′ position of the next by a phosphodiester bridge. In a linear nucleic acid strand, one end typically has a free 5′ phosphate group, the other a free 3′ hydroxyl group. Nucleic acid sequences may be used herein to refer to oligonucleotides, or polynucleotides, and fragments or portions thereof, and to DNA or RNA of genomic or synthetic origin that may be single- or double-stranded, and represent the sense or antisense strand. As used herein, the terms “nucleic acid molecule” and “nucleic acid sequence” will clearly be understood by the skilled person to be interchangeable e.g. when the nucleic acid molecule is comprised within a polynucleotide or vector.

The term “vector”, e.g. an expression vector, as used herein may be a viral vector, bacteriophage, bacterial artificial chromosome, or yeast artificial chromosome. A vector may be a DNA or RNA vector. A vector may be a self-replicating extrachromosomal vector, and aptly, is a DNA plasmid. In some embodiments, the vector means a nucleic acid molecule containing an origin of replication.

When discussing a nucleotide molecule, the terms “upstream” and “downstream” refer to relative positions of genetic code in DNA or RNA. ‘Upstream’ and ‘downstream’ relate to the 5′- to 3′—direction respectively in which RNA transcription takes place. Upstream is toward the 5 end of the RNA molecule and downstream is toward the 3′ end. When considering double-stranded DNA, upstream is toward the 5′ end of the non-template strand for the sequence in question and downstream is toward the 3′ end, whilst the 3′-end of the template strand is upstream of the region in question and the 5′ end is downstream.

Aptly, the expression system may comprise a vector e.g. an expression vector which may further comprise a promoter element. The terms “promoter” and “promoter element” as used herein mean a synthetic or naturally derived molecule which is capable of conferring, activating, or enhancing expression of a nucleic acid in a cell. A promoter element may comprise one or more specific transcriptional regulatory sequences to further enhance expression and/or to alter the spatial expression and/or temporal expression of a gene. A promoter may also comprise distal enhancer or repressor elements, which may be located as much as several thousand base pairs from the start site of transcription. A promoter may regulate the expression of a gene component constitutively, or differentially with respect to the cell, the tissue or organ in which expression occurs or, with respect to the developmental stage at which expression occurs, or in response to external stimuli such as physiological stresses, pathogens, metal ions, or inducing agents. The promoter may be inducible.

In certain embodiments the expression vector may contain more than one promoter (e.g. 2, 3, 4, 5, 6 promoters or more).

In certain embodiments, the promoter may be a eukaryotic promoter. In certain embodiments, the promoter may be selected from a group consisting of EF1a promoter, CMV promoter, SV40 promoter, PGK1 promoter, Ubc promoter, human beta actin promoter, CAG promoter, TRE promoter, UAS promoter, Ac5 promoter, polyhedron promoter, CaMKlla promoter, GAL1 promoter, GAL10 promoter, TEF1 promoter, GDS promoter, ADH1 promoter, ADH1 promoter, CamV35S promoter, Ubi, H1 promoter and U6 promoter.

In certain embodiments, the vector comprises a nucleic acid sequence as set forth in SEQ. ID. No. 6.

In certain embodiments, the promoter may be a prokaryotic promoter (e.g. lac promoter, T7A1 promoter, T7A2 promoter, T7A3 promoter, araBAD promoter, Ptac promoter, pL promoter, Hyper-Spank promoter)

In certain embodiments, the promoter may be a viral promoter (e.g. T7 promoter, SP6 promoter, T3 promoter). In certain embodiments, the expression vector may contain a translation initiation sequence (e.g. a Kozak sequence). In certain embodiments, a translation initiation sequence will be downstream of a promoter.

In certain embodiments, the expression vector contains an internal ribosome entry site (IRES) (e.g. encephalomyocarditis virus IRES, Picornavirus IRES, Apthovirus IRES, Kaposi's sarcoma-associated herpesvirus IRES, Hepatitis A IRES, Hepatitis C IRES, Pestivirus IRES, Cripavirus IRES, Rhopalosiphum padi virus IRES, Marek's disease virus, FGF-1 IRES, FGF-2 IRES, PDGF/c-sis IRES, VEGF IRES, IGF-II IRES, Apaf-1 IRES, Bag-1 IRES, Bcl-2 IRES, BiP IRES, Cat-1 IRES, C-myc IRES, CDK1 IRES, Cyclin D1 IRES, Cyclin T1 IRES, DAP5 IRES, FGF2 IRES, Hiap2 IRES, HIF-1 IRES, IGFR IRES, Mnt IRES, MTG8a IRES, p27 Kip1, p53 IRES, PDGF IRES, PITSLRE IRES, Rev-erb a, UNR IRES, XIAP IRES).

In some embodiments, the IRES is encephalomyocarditis virus IRES.

In certain embodiments, the vector comprises of a nucleic acid sequence as set forth in SEQ. ID. No. 9. In certain embodiments, the expression vector may contain one or more resistance markers e.g. 2 or 3 or more. In certain embodiments the resistance marker may be selected from one or more of Kanamycin, Spectinomycin, Streptomycin, Ampicillin, Carbenicillin, Bleomycin, Erythromycin, Polymyxin B, Tetracycline, Chloramphenicol, Hygromycin, Neomycin, Blasticidin, Puromycin, Geneticin, G418 and Zeiocin.

In certain embodiments, the vector comprises a nucleic acid sequence as set forth in SEQ. ID. No. 13.

In certain embodiments, the vector comprises a nucleic acid sequence as set forth in SEQ. ID. No. 16.

In certain embodiments, the expression vector comprises two nucleic acid sequences as set forth in SEQ. ID. No. 13 and SEQ. ID. No. 16.

In certain embodiments, the expression vector may contain at least one yeast selection marker e.g. 2 or 3 or more. In certain embodiments the resistance marker is selected from HIS3, URA3, LYS2, LEU2, TRP1, MET15, ura4+, leu1+, ade6+ and combinations thereof.

In certain embodiments, the expression vector may contain a transcription termination sequence. In certain embodiments, the expression vector may contain a termination sequence that promotes polyadenylation (e.g. SV40, hGH, BGH, and rbGlob). The polyadenylation may comprise an AAUAAA motif or variant thereof.

In certain embodiments, the expression vector comprises a nucleic acid sequence as set forth in SEQ. ID. No. 11.

In certain embodiments, the method comprises co-expression of a recombinant precursor CFI protein with a recombinant Furin protein. In certain embodiments, both the recombinant precursor CFI protein and the Furin protein are co-expressed from the same expression vector. In certain embodiments, pro-CFI and Furin are expressed from polycistronic RNA.

In certain embodiments, the expression vector is for use in vivo e.g. as a gene therapy. In certain embodiments, the expression system is for use in vivo e.g. as a gene therapy. In certain embodiments, the expression vector comprises a tissue-selective promoter element.

The vector may include nucleic acid sequences that direct autonomous replication in a cell or may include sequences sufficient to allow integration into host cell DNA.

In certain embodiments, the vector is selected from a plasmid (e.g., a DNA plasmid or an RNA plasmid), a cosmid, a bacterial artificial chromosome, and a viral vector.

In certain embodiments, the vector is a viral vector selected from an adenovirus, a replication defective retrovirus, an adeno-associated virus (AAV) and a lentivirus.

As used herein, the term “viral vector” is used to refer either to a nucleic acid molecule (e.g., a transfer plasmid) that includes virus-derived nucleic acid elements that typically facilitate transfer of the nucleic acid molecule or integration into the genome of a cell or to a viral particle that mediates nucleic acid transfer.

Viral particles will typically include various viral components and sometimes also host cell components in addition to nucleic acid(s). The term viral vector may also refer either to a virus or viral particle capable of transferring a nucleic acid into a cell or to the transferred nucleic acid itself. Viral vectors and transfer plasmids contain structural and/or functional genetic elements that are primarily derived from a virus.

Certain embodiments of the invention may relate to infection of cells in order to generate therapeutically significant vectors. Typically, the virus will simply be exposed to the appropriate host cell under physiologic conditions, permitting uptake of the virus.

In certain embodiments, the nucleic acid vector according to the invention is a viral vector, such as a vector derived from an adeno-associated virus, an adenovirus, a retrovirus, a lentivirus, a vaccinia/poxvirus, or a herpesvirus (e.g., herpes simplex virus (HSV)).

In certain embodiments, the vector may be a non-integrating viral vector.

The term “non-integrating viral vector” refers to a vector that typically does not elicit integration of recombinant DNA into the genome of a host cell.

In certain embodiments, the non-integrating nucleic acid vector is an adenovirus vector.

One method for delivery of recombinant DNA into a target cell involves the use of an adenovirus expression vector. Although adenovirus vectors are known to have a low capacity for integration into genomic DNA, this feature is counterbalanced by the high efficiency of gene transfer afforded by these vectors.

As used herein, the term “adenovirus expression vector” refers to constructs containing adenovirus sequences sufficient to (a) support packaging of the construct and (b) to ultimately express a recombinant gene construct that has been cloned therein.

Adenovirus is particularly suitable for use as a gene transfer vector because of its mid-sized genome, ease of manipulation, high titer, wide target-cell range and high infectivity. Both ends of the viral genome contain 100-200 base pair inverted repeats (ITRs), which are cis-elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off. The products of the late genes, including the majority of the viral capsid proteins, are expressed only after significant processing of a single primary transcript issued by the major late promoter (MLP). The MLP is particularly efficient during the late phase of infection, and all the mRNAs issued from this promoter possess a 5-tripartite leader (TPL) sequence which makes them preferred mRNAs for translation.

Generation and propagation of adenovirus vectors, which are replication deficient, depend on a unique helper cell line, designated 293, which was transformed from human embryonic kidney cells (HEK293 cells) by Human Adenovirus 5 DNA fragments and constitutively expresses E1 proteins. Since the E3 region is also dispensable from the adenovirus genome, current adenovirus vectors, with the help of 293 cells, carry foreign DNA in either the E1, the E3 or both regions.

Helper cell lines may be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g., verocells or other monkey embryonic mesenchymal or epithelial cells. As stated above, the preferred helper cell line is HEK293.

Racher et al. (1995) have disclosed improved methods for culturing HEK-293 cells and propagating adenovirus. In one format, natural cell aggregates are grown by inoculating individual cells into 1 litre siliconized spinner flasks (Techne, Cambridge, UK) containing 100-200 ml of medium. Following stirring at 40 rpm, the cell viability is estimated with trypan blue. In another format, Fibra-Cel microcarriers (Bibby Sterlin, Stone, UK) (5 g/I) is employed as follows. A cell inoculum, resuspended in 5 ml of medium, is added to the carrier (50 ml) in a 250 ml Erlenmeyer flask and left stationary, with occasional agitation, for 1 to 4 h. The medium is then replaced with 50 ml of fresh medium and shaking initiated. For virus production, cells are allowed to grow to about 80% confluence, after which time the medium is replaced (to 25% of the final volume) and adenovirus added at a multiplicity of infection of 0.05. Cultures are left stationary overnight, following which the Volume is increased to 100% and shaking commenced for another 72h.

Other than the requirement that the adenovirus vector be replication defective, or at least conditionally defective, the nature of the adenovirus vector is not believed to be crucial to the successful use of the invention. The adenovirus may be of any of the 42 different known serotypes or Subgroups A-F. Adenovirus type 5 of Subgroup C is the preferred starting material in order to obtain the conditional replication-defective adenovirus vector for use in the present invention. This is because adenovirus type 5 is a human adenovirus about which a great deal of biochemical and genetic information is known, and it has historically been used for most constructions employing adenovirus as a vector. As stated above, the typical vector is replication defective and will not have an adenovirus E1 region. Thus, it will be most convenient to introduce the transforming construct at the position from which the E1-coding sequences have been removed. However, the position of insertion of the construct within the adenovirus sequences is not critical. The polynucleotide encoding the gene of interest may also be inserted in lieu of the deleted E3 region in E3 replacement vectors as described by Karlsson et al. (1986) or in the E4 region where a helper cell line or helper virus complements the E4 defect.

Adenovirus growth and manipulation is known to those of skill in the art. Details concerning generation and use of adenovirus vectors can be found in Danthinne, X., Imperiale, M. Production of first-generation adenovirus vectors: a review. Gene Ther 7, 1707-1714 (2000); Nadeau, I., Kamen, A. Biotechnology Advances, Volume 20, Issues 7-8, 2003, Pages 475-489.

This group of viruses can be obtained in high titers, e.g., 10 plaque-forming units per ml, and they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus, demonstrating their safety and therapeutic potential as in vivo gene transfer vectors. Adenovirus vectors have been used in eukaryotic gene expression and vaccine development. Adenoviral vectors also have been described for treatment of certain types of cancers (U.S. Pat. No. 5,789,244). Recombinant adenovirus could be used for gene therapy.

As described above, methods for the production of adenoviral vectors for gene therapy are well known in the art.

In certain embodiments, the vector may be an integrating viral vector.

The term “integrating viral vector” refers to a vector that typically elicits integration of recombinant DNA into the genome of a host cell.

In preferred embodiments, the integrating nucleic acid vector is an adeno-associated virus vector.

Adeno-associated virus (AAV) is an attractive vector system for use in certain embodiments of the present invention as it has a high frequency of integration and it can infect non-dividing cells. AAV has a broad host range for infectivity, and well characterised functional mechanisms.

Details concerning the generation and use of recombinant AAV vectors can be found in U.S. Pat. Nos. 5,139,941 and 4,797,368.

The genomic organization of all known AAV serotypes is very similar. The genome of AAV is a linear, single-stranded DNA molecule that is less than about 5,000 nucleotides (nt) in length. Inverted terminal repeats (ITRs) flank the unique coding nucleotide sequences for the non-structural replication (Rep) proteins and the structural (VP) proteins. The VP proteins (VP1, -2 and -3) form the capsid or protein shell. The terminal 145 nt are self-complementary and are organized so that an energetically stable intramolecular duplex forming a T-shaped hairpin may be formed. These hairpin structures function as an origin for viral DNA replication, serving as primers for the cellular DNA polymerase complex. Following wild type AAV infection in mammalian cells the Rep genes 25 (i.e. Rep78 and Rep52) are expressed from the P5 promoter and the PI 9 promoter, respectively and both Rep proteins have a function in the replication of the viral genome. A splicing event in the Rep ORF results in the expression of actually four Rep proteins (i.e. Rep78, Rep68, Rep52 and Rep40). However, it has been shown that the unspliced mRNA, encoding Rep78 and Rep52 proteins, in mammalian cells are sufficient for AAV vector production. AAV infection in mammalian cells relies for the capsid proteins production on a combination of alternate usage of two splice acceptor sites and the suboptimal utilization of an ACG initiation codon for VP2.

A recombinant AAV-transgene vector (rAAV) may have one or preferably all wild type AAV genes deleted, but may still comprise functional ITR nucleic acid sequences. The rAAV-transgene vector may not comprise any nucleotide sequences encoding viral proteins, such as the rep (replication) or cap (capsid) genes of AAV. Functional ITR sequences are necessary for the replication, rescue and packaging of AAV virions. The ITR sequences may be wild type sequences or may have at least 80%, 85%, 90%>, 95%, or 100% sequence identity with wild type sequences or may be altered by for example in insertion, mutation, deletion or substitution of nucleotides, as long as they remain functional. In this context, functionality refers to the ability to direct packaging of the genome into the capsid shell and then allow for expression in the host cell to be transduced or target cell. Typically, the inverted terminal repeats of the wild type AAV genome are retained in the rAAV-transgene vector. The ITRs can be cloned from the AAV viral genome or excised from a vector comprising the AAV ITRs. The ITR nucleotide sequences can be either ligated at either end to a transgene as defined herein using standard molecular biology techniques, or the wild type AAV sequence between the ITRs can be replaced with the desired nucleotide sequence. The rAAV-transgene vector may comprise at least the nucleotide sequences of the inverted terminal repeat regions (ITR) of one of the AAV serotypes, or nucleotide sequences substantially identical thereto, and at least one nucleotide sequence encoding a therapeutic protein (under control of a suitable regulatory element) inserted between the two ITRs. The majority of currently used rAAV- transgene vectors use the ITR sequences from AAV serotype 2.

In some embodiments, the AAV ITRs are AAV2 or AAV8 ITRs. In preferred embodiments, the AAV ITRs are AAV2 ITRs.

Multiple serotypes of adeno-associated virus (AAV), including 12 human serotypes (AAV1, AAV2, AAV3, AAV4, AAV5, AAV6, AAV7, AAV8, AAV9, AAV10, AAV1 1, and AAV12) and more than 100 serotypes from nonhuman primates have now been identified. Howarth et al., Using viral vectors as gene transfer tools. Cell Biol. Toxicol. 26: 1-10 (2010). The serotype of the inverted terminal repeats (ITRs) or the capsid sequence of the AAV vector may be selected from any known human or nonhuman AAV serotype. The production, purification, and characterization of the recombinant AAV vectors of embodiments of present invention may be carried out using any of the many methods known in the art. For reviews of laboratory-scale production methods, see, e.g., Clark, Recent advances in recombinant adeno-associated virus vector production. Kidney Int. 61s:9-15 (2002).

In certain embodiments, the expression system comprises an AAV derived nucleic acid vector encoding inverted terminal repeats (ITRs) flanking the desired expression cassette. The vector may comprise at least one ITR adjacent to said expression cassette promoter (L-ITR) at the first end of the expression cassette, and at least one ITR adjacent to the polyadenylation sequence (R-ITR) at the second end of the expression cassette opposite to the first end.

“Flanking” means that the ITRs are located at both sides of the transgene expression cassette, i.e. at the 5 and 3′ termini. The ITRs thereby frame the transgene expression cassette.

The viral vector, such as an AAV vector, may comprise any suitable promoter, the selection of which may be readily made by the skilled person. The promoter sequence may be constitutively active (i.e. operational in any host cell background), or alternatively may be active only in a specific host cell environment, thus allowing for targeted expression of the transgene in a particular cell type (e.g. a tissue-specific promoter). Where the vector is administered for therapy, it is preferred that the promoter should be functional in the target cell.

Preferred promoters, which are not retinal-cell specific, include the chicken beta-actin (CBA) promoter, optionally in combination with a cytomegalovirus (CMV) enhancer element. An example promoter for use in the invention is a CAG promoter.

An example CMV promoter sequence is:

(SEQ ID NO: 17)
GGAGTTCCGCGTTACATAACTTACGGTAAATGGCCCGCCTGGCTGACCGC
CCAACGACCCCCGCCCATTGACGTCAATAATGACGTATGTTCCCATAGTA
ACGCCAATAGGGACTTTCCATTGACGTCAATGGGTGGAGTATTTACGGTA
AACTGCCCACTTGGCAGTACATCAAGTGTATCATATGCCAAGTACGCCCC
CTATTGACGTCAATGACGGTAAATGGCCCGCCTGGCATTATGCCCAGTAC
ATGACCTTATGGGACTTTCCTACTTGGCAGTACATCTACGTATTAGTCAT
CGCTATTACCATGGTGATGCGGTTTTGGCAGTACATCAATGGGCGTGGAT
AGCGGTTTGACTCACGGGGATTTCCAAGTCTCCACCCCATTGACGTCAAT
GGGAGTTTGTTTTGGCACCAAAATCAACGGGACTTTCCAAAATGTCGTAA
CAACTCCGCCCCATTGACGCAAATGGGCGGTAGGCGTGTACGGTGGGAGG
TCTATATAAGCAGAGCTCGTTTAGTGAACCGTCAGATCGCCTGGAGACGC
CATCCACGCTGTTTTGACCTCCATAGAAGACACCG

In some embodiments, the viral vector comprises a promoter with a nucleotide sequence that has at least 75%, 80%, 85% 90%, 95%, 96%, 97%, 98% or 99% identity to SEQ ID NO: 17. Preferably, wherein the nucleotide sequence substantially retains the functional activity of the promoter represented by SEQ ID NO: 17.

An example CAG promoter sequence is:

(SEQ ID NO: 18)
CCATTGACGTCAATAATGACGTATGTTCCCATAGTAACGCCAATAGGGAC
TTTCCATTGACGTCAATGGGTGGAGTATTTACGGTAAACTGCCCACTTGG
CAGTACATCAAGTGTATCATATGCCAAGTACGCCCCCTATTGACGTCAAT
GACGGTAAATGGCCCGCCTGGCATTATGCCCAGTACATGACCTTATGGGA
CTTTCCTACTTGGCAGTACATCTACGTATTAGTCATCGCTATTACCATGG
TCGAGGTGAGCCCCACGTTCTGCTTCACTCTCCCCATCTCCCCCCCCTCC
CCACCCCCAATTTTGTATTTATTTATTTTTTAATTATTTTGTGCAGCGAT
GGGGGCGGGGGGGGGGGGGGGGCGCGCGCCAGGCGGGGCGGGGCGGGGCG
AGGGGCGGGGCGGGGCGAGGCGGAGAGGTGCGGCGGCAGCCAATCAGAGC
GGCGCGCTCCGAAAGTTTCCTTTTATGGCGAGGCGGCGGCGGCGGCGGCC
CTATAAAAAGCGAAGCGCGCGGCGGGCGGGAGTCGCTGCGCGCTGCCTTC
GCCCCGTGCCCCGCTCCGCCGCCGCCTCGCGCCGCCCGCCCCGGCTCTGA
CTGACCGCGTTACTCCCACAGGTGAGCGGGCGGGACGGCCCTTCTCCTCC
GGGCTGTAATTAGCGCTTGGTTTAATGACGGCTTGTTTCTTTTCTGTGGC
TGCGTGAAAGCCTTGAGGGGCTCCGGGAGGGCCCTTTGTGCGGGGGGAGC
GGCTCGGGGCTGTCCGCGGGGGGACGGCTGCCTTCGGGGGGGACGGGGCA
GGGCGGGGTTCGGCTTCTGGCGTGTGACCGGCGGCTCTAGAGCCTCTGCT
AACCATGTTCATGCCTTCTTCTTTTTCCTACAGCTCCTGGGCAACGTGCT
GGTTATTGTGCTGTCTCATCATTTTGGCAAAGAATTGGATCC

In some embodiments, the viral vector comprises a promoter with a nucleotide sequence that has at least 75%, 80%, 85% 90%, 95%, 96%, 97%, 98% or 99% identity to SEQ ID NO: 18. Preferably, the nucleotide sequence substantially retains the functional activity of the promoter represented by SEQ ID NO: 18.

In other embodiments, the viral vector comprises a promoter with the nucleotide sequence of SEQ ID NO: 18.

Preferably, the promoter is upstream of the nucleic acid molecule encoding the precursor Complement Factor I protein or variant thereof and the nucleic acid molecule encoding the furin protein or variant thereof.

In some embodiments, the viral vector further comprises a nucleic acid molecule encoding an internal ribosome entry site (IRES), preferably wherein the nucleic acid molecule encoding IRES is positioned between the nucleic acid molecule or variant thereof encoding the precursor Complement Factor I protein or variant thereof and the nucleic acid molecule encoding the furin protein or variant thereof.

In some embodiments, the viral vector further comprises a nucleotide sequence encoding a post-transcriptional regulatory element. Preferably, the nucleotide sequence encodes a woodchuck hepatitis post-transcriptional regulatory element (WPRE) regulatory element or variant thereof. Preferably, the WPRE regulatory element is downstream of the nucleic acid molecule encoding the precursor Complement Factor I protein or variant thereof and the nucleic acid molecule encoding the furin protein or variant thereof.

An example WPRE is

(SEQ ID NO: 19)
AATCAACCTCTGGATTACAAAATTTGTGAAAGATTGACTGGTATTCTTAA
CTATGTTGCTCCTTTTACGCTATGTGGATACGCTGCTTTAATGCCTTTGT
ATCATGCTATTGCTTCCCGTATGGCTTTCATTTTCTCCTCCTTGTATAAA
TCCTGGTTGCTGTCTCTTTATGAGGAGTTGTGGCCCGTTGTCAGGCAACG
TGGCGTGGTGTGCACTGTGTTTGCTGACGCAACCCCCACTGGTTGGGGCA
TTGCCACCACCTGTCAGCTCCTTTCCGGGACTTTCGCTTTCCCCCTCCCT
ATTGCCACGGCGGAACTCATCGCCGCCTGCCTTGCCCGCTGCTGGACAGG
GGCTCGGCTGTTGGGCACTGACAATTCCGTGGTGTTGTCGGGGAAATCAT
CGTCCTTTCCTTGGCTGCTCGCCTGTGTTGCCACCTGGATTCTGCGCGGG
ACGTCCTTCTGCTACGTCCCTTCGGCCCTCAATCCAGCGGACCTTCCTTC
CCGCGGCCTGCTGCCGGCTCTGCGGCCTCTTCCGCGTCTTCGCCTTCGCC
CTCAGACGAGTCGGATCTCCCTTTGGGCCGCCTCCCCGC

A shortened version of WPRE, which contains only minimal gamma and alpha elements (referred to as WPRE3; Choi, J.-H. et al. (2014) Molecular Brain 7: 17), may also be used in the invention. An example WPRE3 sequence is:

(SEQ ID NO: 20)
AATCAACCTCTGGATTACAAAATTTGTGAAAGATTGACTGGTATTCTTAA
CTATGTTGCTCCTTTTACGCTATGTGGATACGCTGCTTTAATGCCTTTGT
ATCATGCTATTGCTTCCCGTATGGCTTTCATTTTCTCCTCCTTGTATAAA
TCCTGGTTAGTTCTTGCCACGGCGGAACTCATCGCCGCCTGCCTTGCCCG
CTGCTGGACAGGGGCTCGGCTGTTGGGCACTGACAATTCCGTGGT

In some embodiments, the viral vector comprises a post-transcriptional regulatory element with a nucleotide sequence that has at least 75%, 80%, 85%, 90%, 95%, 96%, 97%, 98% or 99% identity to SEQ ID NO: 19 or 20. Preferably, wherein the nucleotide sequence substantially retains the functional activity of the post-transcriptional regulatory element represented by SEQ ID NO: 19 or 20.

In other embodiments, the viral vector comprises a post-transcriptional regulatory element with the nucleotide sequence of SEQ ID NO: 19 or 20.

In some embodiments, the viral vector further comprises a nucleotide sequence encoding a poly-A signal. Preferably, the poly-A signal is downstream of the nucleic acid molecule encoding the precursor Complement Factor I protein or variant thereof and the nucleic acid molecule encoding the furin protein or variant thereof.

A preferred polyadenylation site is the Bovine Growth Hormone poly-A (bGH poly-A) signal. Therefore, in some embodiments, the viral vector comprises a nucleotide sequence encoding a Bovine Growth Hormone poly-A signal. Preferably, the Bovine Growth Hormone poly-A signal is downstream of the nucleic acid molecule encoding the precursor Complement Factor I protein or variant thereof and the nucleic acid molecule encoding the furin protein or variant thereof.

An example Bovine Growth Hormone poly-A (bGH poly-A) signal is:

(SEQ ID NO: 21)
CGACTGTGCCTTCTAGTTGCCAGCCATCTGTTGTTTGCCCCTCCCCCGTG
CCTTCCTTGACCCTGGAAGGTGCCACTCCCACTGTCCTTTCCTAATAAAA
TGAGGAAATTGCATCGCATTGTCTGAGTAGGTGTCATTCTATTCTGGGGG
GTGGGGTGGGGCAGGACAGCAAGGGGGAGGATTGGGAAGACAATAGCAGG
CATGCTGGGGATGCGGTGGGCTCTATGG

A further example Bovine Growth Hormone poly-A (bGH poly-A) signal is:

(SEQ ID NO: 22)
TCGCTGATCAGCCTCGACTGTGCCTTCTAGTTGCCAGCCATCTGTTGTTT
GCCCCTCCCCCGTGCCTTCCTTGACCCTGGAAGGTGCCACTCCCACTGTC
CTTTCCTAATAAAATGAGGAAATTGCATCGCATTGTCTGAGTAGGTGTCA
TTCTATTCTGGGGGGTGGGGTGGGGCAGGACAGCAAGGGGGAGGATTGGG
AAGACAATAGCAGGCATGCTGGGGATGCGGTGGGCTCTATGGCTTCTGAG
GCGGAAAGAACCAGCTGGGG

In some embodiments, the viral vector comprises a polyadenylation signal with a nucleotide sequence that has at least 75%, 80%, 85%, 90%, 95%, 96%, 97%, 98% or 99% identity to SEQ ID NO: 21 or 22. Preferably, wherein the nucleotide sequence substantially retains the functional activity of the polyadenylation signal represented by SEQ ID NO: 21 or 22.

In other embodiments, the viral vector comprises a polyadenylation signal with the nucleotide sequence of SEQ ID NO: 21 or 22.

In some embodiments, the viral vector comprises:

(a) a 5′ AAV ITR;

(b) a CMV promoter or a CAG promoter;

(c) a nucleic acid molecule encoding the precursor Complement Factor I protein or variant thereof;

(d) a nucleic acid molecule encoding an IRES site or a self cleaving peptide e.g., 2A sequence;

(e) a nucleic acid molecule encoding the furin protein or variant thereof

(f) a poly-A signal, preferably a Bovine Growth Hormone poly-A signal; and

(g) a 3′ AAV ITR.

In some embodiments, the viral vector comprises:

(a) a 5′ AAV ITR;

(b) a CMV promoter or a CAG promoter;

(c) a nucleic acid molecule encoding the precursor Complement Factor I protein or variant thereof;

(d) a nucleic acid molecule encoding an IRES site or a self cleaving peptide e.g., 2A sequence;

(e) a nucleic acid molecule encoding the furin protein or variant thereof;

(f) a WPRE regulatory element, optionally wherein the WPRE regulatory element is a WPRE3 regulatory element;

(g) a poly-A signal, preferably a Bovine Growth Hormone poly-A signal; and

(h) a 3′ AAV ITR.

Suitably the nucleic acid molecules defined by (c) and (e) may be in the reciprocal position in the vector.

In some embodiments, the viral vector comprises:

(a) a 5′ AAV ITR;

(b) a CMV promoter or a CAG promoter;

(c) a nucleic acid molecule encoding the furin protein or variant thereof;

(d) a nucleic acid molecule encoding an IRES site or a self cleaving peptide e.g., 2A sequence;

(e) a nucleic acid molecule encoding the precursor Complement Factor I protein or variant thereof;

(f) a poly-A signal, preferably a Bovine Growth Hormone poly-A signal; and

(g) a 3′ AAV ITR.

In some embodiments, the viral vector comprises:

(a) a 5′ AAV ITR;

(b) a CMV promoter or a CAG promoter;

(c) a nucleic acid molecule encoding the furin protein or variant thereof;

(d) a nucleic acid molecule encoding an IRES site or a self cleaving peptide e.g., 2A sequence;

(e) a nucleic acid molecule encoding the precursor Complement Factor I protein or variant thereof;

(f) a WPRE regulatory element, optionally wherein the WPRE regulatory element is a WPRE3 regulatory element;

(g) a poly-A signal, preferably a Bovine Growth Hormone poly-A signal; and

(h) a 3′ AAV ITR.

Suitably, in embodiments where (b) comprises a CAG promoter; (f) may be a WPRE3 regulatory element.

The viral vector may comprise (a)-(g) or (a)-(h) in order, from 5′ to 3′.

In certain embodiments, the viral vector is an adeno-associated virus (AAV), adenovirus or a lentivirus, most preferably an AAV vector.

In some embodiments, the AAV vector is in the form of an AAV particle.

Methods of preparing and modifying viral vectors and viral vector particles, such as those derived from AAV, are well known in the art. In one embodiment of the invention, the AAV vector particle is an AAV1, AAV2, AAV3, AAV4, AAV5, AAV6, AAV7, AAV8, AAV9, AAV10, AAV11, Rec2 or Rec3 AAV vector particle.

In one embodiment the AAV may be an AAV1, AAV2, AAV5, AAV7, AAV8 or AAV8 serotype.

In one embodiment the AAV may be an AAV2 or AAV8 serotype.

The tropism of AAVs may be refined by combining capsids and genomes from different serotypes. The AAV particles of the invention may include transcapsidated forms wherein an AAV genome or derivative having an ITR of one serotype is packaged in the capsid of a different serotype. The AAV particles of the invention also include mosaic forms wherein a mixture of unmodified capsid proteins from two or more different serotypes makes up the viral capsid. Chimeric, shuffled or capsid-modified derivatives may be selected to provide one or more desired functionalities for the AAV vector.

For example, ITRs from any one of serotypes AAV1-13 may be retained and packaged into capsids of a different serotype, for example an AAV capsid selected from any one of AAV1-12, AAV7m8, AAV-DJ, AAV-DJ8, AAV-DJ9, AAVrh8, AAVrh8R, AAVrh10, AAVrh39, AAVRec2 or AAVRec3.

In some embodiments, the ITRs of AAV2 are retained and packaged into capsids of a different serotype.

The serotype to be used in the invention can be selected according to the route of delivery and target cell to be transduced.

The AAV serotype determines the tissue specificity of infection (or tropism) of an AAV virus. Accordingly, preferred AAV serotypes for use in AAVs administered to patients in accordance with the invention are those which have natural tropism for or a high efficiency of infection of target cells, preferably target cells within the eye. In one embodiment, AAV serotypes for use in the invention are those which transduce cells of the neurosensory retina, retinal pigment epithelium and/or choroid.

In some embodiments, the AAV particles of the invention include those with an AAV2 genome and AAV2 capsid proteins (AAV2/2), those with an AAV2 genome and AAV5 capsid proteins (AAV2/5) and those with an AAV2 genome and AAV8 capsid proteins (AAV2/8).

Preferably the viral vector or viral vector particle is for use as a medicament, preferably for use in the treatment of a complement mediated disorder.

An example wild type nucleotide sequence encoding Complement Factor I is disclosed herein as SEQ ID NO: 10.

In some embodiments, the nucleotide sequence encoding Complement Factor I has at least 75%, 80%, 85%, 86%, 87%, 88%, 89%, 90%, 91%, 92%, 93%, 94%, 95%, 96%, 97%, 98% or 99% identity to SEQ ID NO: 10. Preferably, the protein encoded by the nucleotide sequence substantially retains a functional activity of the protein represented by SEQ ID NO: 1.

In other embodiments, the nucleotide sequence encoding Complement Factor I is SEQ ID NO: 10.

In other embodiments, the nucleotide sequence encoding Complement Factor I has at least 75%, 80%, 85%, 90%, 91%, 92%, 93%, 94%, 95%, 96%, 97%, 98% or 99% identity to positions 55 to 1752 of SEQ ID NO: 10. Preferably, the protein encoded by the nucleotide sequence substantially retains a functional activity of the protein represented by SEQ ID NO: 1.

In other embodiments, the nucleotide sequence encoding Complement Factor I is positions 55 to 1752 of SEQ ID NO: 10.

In other embodiments, the nucleotide sequence encoding Complement Factor I encodes an amino acid sequence that has at least 75%, 80%, 85% 90%, 95%, 96%, 97%, 98% or 99% identity to SEQ ID NO: 1. Preferably, wherein the amino acid sequence substantially retains a functional activity of the protein represented by SEQ ID NO: 1.

In other embodiments, the nucleotide sequence encoding Complement Factor I encodes the amino acid sequence SEQ ID NO: 1.

In other embodiment, the nucleotide sequence encoding Complement Factor I encodes an amino acid sequence that has at least 75%, 80%, 85%, 90%, 95%, 96%, 97%, 98%, 99% identity to positions 19 to 583 of SEQ ID NO: 1. Preferably, wherein the amino acid sequence substantially retains a functional activity of the protein represented by SEQ ID NO: 1.

In other embodiments, the nucleotide sequence encoding Complement Factor I encodes the amino acid sequence of positions 19 to 583 of SEQ ID NO: 1.

In a further aspect of the present invention, there is provided a method for producing a recombinant mature Complement Factor I protein or variant thereof, the method comprising expressing a recombinant precursor Complement Factor I protein or variant thereof and a recombinant furin protein or variant thereof in a host cell under conditions suitable for the expressed furin protein to cleave the expressed recombinant precursor Complement Factor 1 protein or variant thereof to form the recombinant mature Complement Factor I protein or variant thereof.

In certain embodiments, the method comprises transforming the cell with a nucleic acid molecule encoding a precursor complement factor I protein. Aptly, the method comprises transforming the cell with a vector which encodes a precursor complement system protein or encodes precursor complement system protein and a protease enzyme (e.g Furin) as described herein. In certain embodiments, a precursor complement system protein and protease enzyme are co-expressed from the same nucleic acid molecule. In certain embodiments, a precursor complement system and protease enzyme are co-expressed from a polycistronic RNA.

Aptly, the method comprises transforming the cell with at least one vector which encodes a precursor CFI protein, or Furin, or encodes precursor CFI protein and a protease enzyme (e.g. Furin) as described herein.

In certain embodiments, a precursor-CFI and protease enzyme (e.g. Furin) are co-expressed from the same nucleic acid molecule. In certain embodiments, a precursor-CFI and protease enzyme are co-expressed from a polycistronic RNA.

In an aspect of the present invention, there is provided use of an expression system as defined herein in a method of gene therapy wherein the expression vectors may include one or more sequences that direct autonomous replication in a cell and/or may include sequences sufficient to allow integration into host cell DNA.

In certain embodiments, the invention provides a method of gene therapy for a subject wherein the subject suffers from a complement system mediated disorder.

In certain embodiments, the invention provides an expression system and/or expression vector wherein the expression system and/or vector increases the concentration of mature Complement Factor I within a human subject.

In an aspect of the present invention, there is provided a method of treating and/or preventing a complement system-mediated disorder in a subject, wherein the method comprises administering the expression system and/or the expression vector of certain aspects of the invention to a subject in need thereof.

In certain embodiments, the method is for the treatment and/or prevention of a disorder associated with a mutation in a CFI protein.

In certain embodiments, the method is for the treatment and/or prevention of a complement mediated disorder selected from atypical haemolytic uremic syndrome (aHUS); membranoproliferative glomerulonephritis Type 2 (MPGN2); Huntingdon's disease; Guillain-Barr6 syndrome, Multiple sclerosis (MS), Alzheimer's disease, Parkinson's disease, allergic encephalomyelitis, Myasthenia gravis (MG); systemic lupus erythematosus, systemic sclerosis, rheumatoid arthritis; cardiovascular diseases or conditions such as myocardial infarction, chronic cardiovascular disease, atherosclerosis or stroke; haematological disorders such as paroxysmal nocturnal haemoglobinuria; respiratory disorder such as asthma; dermatological diseases such as bullous pemphigoid or psoriasis; treatment following organ transplant rejection, graft versus host disease; ocular diseases or conditions such as age-related macular degeneration (AMD), glaucoma, diabetic retinopathy, early-onset macular degeneration, Stargardt's disease, central serous chorioretinopathy or retinitis pigmentosa, neuromyelitis optica or uveitis; or other inflammatory and/or autoimmune diseases.

In certain embodiments, the method is for the treatment of CFI protein deficiency. In certain embodiments, the method is for the treatment of a disorder associated with CFI protein deficiency selected from glomerulonephritis associated with C3 deposition, rheumatoid arthritis and systemic lupus erythematosus (SLE).

In preferred embodiments, the disorder is a complement mediated ocular disorder or disease such as age-related macular degeneration (AMD), glaucoma, diabetic retinopathy, early-onset macular degeneration, Stargardt's disease, central serous chorioretinopathy or retinitis pigmentosa, neuromyelitis optica or uveitis. Most preferably, the disorder is AMD.

In certain embodiments, the method comprises administering an expression vector of certain aspects of the invention to a subject. In certain embodiments, the recombinant mature CFI protein expressed by the expression vector is for use in therapy. In certain embodiments, the method comprises administering the expression vector to a region of an eye of a subject. In certain embodiments, the method comprises injecting an expression vector of certain aspects of the invention into the subject's eye. In certain embodiments, administration to the eye is by subretinal, suprachoroidal or intravitreal injection. Aptly, the method is for the treatment of a complement mediated ocular disorder or disease such as age-related macular degeneration (AMD), glaucoma, diabetic retinopathy, early-onset macular degeneration, Stargardt's disease, central serous chorioretinopathy or retinitis pigmentosa, neuromyelitis optica or uveitis. Preferably, the disorder is age-related macular degeneration.

In certain embodiments, the method comprises administering an expression system of certain aspects of the invention to a subject. In certain embodiments, the recombinant mature CFI protein expressed by the expression system is for use in therapy. In certain embodiments, the method comprises administering the expression system to a region of an eye of a subject. In certain embodiments, the method comprises injecting an expression system of certain aspects of the invention into the subject's eye. In certain embodiments, administration to the eye is by subretinal, suprachoroidal or intravitreal injection. Aptly, the method is for the treatment of a complement mediated ocular disorder or disease such as age-related macular degeneration (AMD), glaucoma, diabetic retinopathy, early-onset macular degeneration, Stargardt's disease, central serous chorioretinopathy or retinitis pigmentosa, neuromyelitis optica or uveitis. Preferably, the disorder is age-related macular degeneration.

In some embodiments, the formation of geographic atrophy is prevented or reduced, and/or the amount of geographic atrophy is reduced.

In some embodiments, the progression of geographic atrophy is slowed.

In some embodiments, there is at least a 10% reduction in the increase in geographic atrophy area over the 12 months following administration to a treated eye of a subject, relative to an untreated eye over the same period. In other embodiments, there is at least 20%, 30%, 40%, 50%, 60%, 70%, 80% or 90% reduction in the increase in geographic atrophy area over the 12 months following administration to a treated eye of a subject, relative to an untreated eye over the same period.

In some embodiments, the level of C3b-inactivating and iC3b-degradationactivity is increased in a subject, or in an eye, such as in the retinal pigment epithelium (RPE) aqueous humor and/or vitreous humor, of a subject, optionally to a level that exceeds a normal level in a subject, or eye or RPE thereof

In some embodiments, the use is for treating or preventing a disorder in a subject: (a) having a normal level of Complement Factor I activity or concentration in the eye and/or serum, preferably at least 30 pg/mL, such as 30-40 pg/mL in serum; and/or

(b) not carrying a rare Complement Factor I variant allele.

In certain embodiments, the method comprises administering the expression vector of certain aspects of the present invention via oral administration, parental administration and/or via inhalation. In certain embodiments, the method comprises administering the recombinant mature CFI protein expressed by the expression vector via oral administration, parental administration and/or via inhalation.

In certain embodiments, the method comprises administering the expression system of certain aspects of the present invention via oral administration, parental administration and/or via inhalation. In certain embodiments, the method comprises administering the recombinant mature CFI protein expressed by the expression system via oral administration, parental administration and/or via inhalation.

The expression vector, system and/or the recombinant mature CFI protein may be administered in a composition.

Accordingly, in certain embodiments there is provided a composition comprising the expression vector, system and/or the recombinant mature CFI protein.

The expression vector, system and/or the recombinant mature CFI protein, or composition comprising the same, may be for administration to a subject in need thereof e.g. a subject suffering from a complement system mediated disorder.

The composition may be for oral administration and/or parental administration. The composition may be for administration in the eye of a subject.

The precise nature of the carrier or other material may be determined by the skilled person according to the route of administration, e.g. subretinal, suprachoroidal or intravitreal injection.

Compositions of certain embodiments of the invention may be administered with or without an excipient. Excipients include, for example, encapsulating materials or additives such as absorption accelerators, antioxidants, binders, buffers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents and mixtures thereof. The excipients may be pharmaceutically acceptable excipients.

The pharmaceutical composition is typically in liquid form. Liquid pharmaceutical compositions generally include a liquid carrier such as water, petroleum, animal or vegetable oils, mineral oil or synthetic oil. Physiological saline solution, magnesium chloride, dextrose or other saccharide solution, or glycols such as ethylene glycol, propylene glycol or polyethylene glycol may be included. In some cases, a surfactant, such as pluronic acid (PF68) 0.001% may be used.

Excipients for preparation of compositions comprising a compositions of certain embodiments to be administered orally in solid dosage form include, for example, agar, alginic acid, aluminum hydroxide, benzyl alcohol, benzyl benzoate, 1,3-butylene glycol, carbomers, castor oil, cellulose, cellulose acetate, cocoa butter, corn starch, corn oil, cottonseed oil, cross-povidone, diglycerides, ethanol, ethyl cellulose, ethyl laureate, ethyl oleate, fatty acid esters, gelatin, germ oil, glucose, glycerol, groundnut oil, hydroxypropylmethyl cellulose, isopropanol, isotonic saline, lactose, magnesium hydroxide, magnesium stearate, malt, mannitol, monoglycerides, olive oil, peanut oil, potassium phosphate salts, potato starch, povidone, propylene glycol, Ringer's solution, safflower oil, sesame oil, sodium carboxymethyl cellulose, sodium phosphate salts, sodium lauryl sulfate, sodium sorbitol, soybean oil, stearic acids, stearyl fumarate, sucrose, surfactants, talc, tragacanth, tetrahydrofurfuryl alcohol, triglycerides, water, and mixtures thereof.

Excipients for preparation of compositions of certain embodiments of the present invention to be administered orally in liquid dosage forms include, for example, 1,3-butylene glycol, castor oil, corn oil, cottonseed oil, ethanol, fatty acid esters of sorbitan, germ oil, groundnut oil, glycerol, isopropanol, olive oil, polyethylene glycols, propylene glycol, sesame oil, water and mixtures thereof.

Excipients for preparation of compositions of certain embodiments of the present invention to be administered osmotically include, for example, chlorofluorohydrocarbons, ethanol, water and mixtures thereof. Excipients for preparation of compositions of certain embodiments of the present invention to be administered parenterally include, for example, 1,3-butanediol, castor oil, corn oil, cottonseed oil, dextrose, germ oil, groundnut oil, liposomes, oleic acid, olive oil, peanut oil, Ringer's solution, safflower oil, sesame oil, soybean oil, U.S.P. or isotonic sodium chloride solution, water and mixtures thereof.

Excipients for preparation of compositions comprising a compound of this invention to be administered rectally or vaginally include, for example, cocoa butter, polyethylene glycol, wax and mixtures thereof.

Aptly, the expression vector, system and/or the recombinant mature CFI protein is for administration in a therapeutically effective amount. As used herein, the term “therapeutically effective amount” ” is taken to refer to an amount of an agent, e.g. an agent of certain embodiments that produces a desired therapeutic effect in the patient, such as preventing or treating a target condition or alleviating symptoms associated with the condition. Aptly, the precise therapeutically effective amount is an amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. A person skilled in the clinical and pharmacological arts will be able to determine a therapeutically effective amount of the composition to administer through routine experimentation, namely by monitoring a patient's response to administration of a compound and adjusting the dosage accordingly. For additional guidance, see Remington: The Science and Practice of Pharmacy 21st Edition, Univ. of Sciences in Philadelphia (USIP), Lippincott Williams & Wilkins, Philadelphia, Pa., 2005.

In some embodiments, the expression vector or expression system of the invention may be formulated into a pharmaceutical composition for subretinal, suprachoroidal or intravitreal injection. The volume of the pharmaceutical composition injected may, for example, be about in a volume of about 10-500 pL, for example about 50-500, 100-500, 200-500, 300-500, 400-500, 50-250, 100-250, 200-250 or 50-150 μL. The volume may, for example, be about 10, 50, 100, 150, 200, 250, 300, 350, 400, 450 or 500 μL. Preferably, the volume is 100 μL.

The skilled person will be familiar with and well able to carry out individual subretinal, direct retinal, suprachoroidal or intravitreal injections.

In one embodiment described herein, the expression vector or expression system, or pharmaceutical composition comprising the same, is administered not more than once, or not more than twice, during the lifetime of a subject.

In some embodiments, the pharmaceutical composition administered by subretinal, suprachoroidal or intravitreal injection may be administered at a dose of at least 1e8 or 1e9 vector genomes [vg] per eye, for example at a dose of 1e8 vg/eye, 2e8 vg/eye, 1e9 vg/eye, 2e9vg/eye, 1e10 vg/eye, 2e10vg/eye, 5e10 vg/eye, 1e11 vg/eye, 2e11 vg/eye, 5e11 vg/eye, or 1e12 vg/eye.

In a further aspect of the present invention there is provided a method of isolating recombinant mature CFI protein from one or more cell components. In certain embodiments, the method is for separating recombinant mature CFI protein from recombinant precursor CFI protein. In certain embodiments, the method involves contacting a preparation containing recombinant precursor and mature complement system protein and optionally one or more further with a chromatography material under conditions such that the precursor and mature form CFI protein are bound to said material. In certain embodiments the chromatography material comprises:

a) a strong cation exchange chromatography resin (e.g., sulfonated polystyrene);

b) a weak cation exchange chromatography resin (e.g. carboxylic methacrylate);

c) a strong anion exchange chromatography resin (e.g. quaternary ammonium polystyrene);

d) a weak anion exchange chromatography resin (e.g. polyamine, polystyrene, or phenol);

e) protein A affinity chromatography resin;

f) protein G affinity chromatography resin;

g) protein A/G affinity chromatography resin;

h) protein L affinity chromatography resin;

i) immobilized metal affinity chromatography resin (e.g. Nickel-NTA resin or Cobalt-NTA resin);

j) Glutathione S-transferase (GST) affinity chromatography resin,

k) a hydrophobic interaction resin (e.g. butyl resin or octyl resin),

I) OX21 monoclonal antibody—NHS-Sepharose), and/or

m) size exclusion chromatography resin.

In certain embodiments, the method involves contacting a preparation comprising recombinant precursor and mature complement system protein and optionally one more cellular components with at least one further chromatography material (e.g. contacting the preparation with one, two, three, or more chromatography materials).

In certain embodiments, the method comprises contacting a preparation containing precursor and mature form complement factor I proteins with at least one further chromatographic material in order to obtain a series of distinct eluates wherein the precursor complement system protein and mature form complement system protein are substantially separated from one another, and/or wherein the precursor complement factor I protein and mature form complement factor I protein are substantially separated from other cellular components.

In certain embodiments, the method comprises contacting a preparation containing precursor and mature form complement factor I proteins with an affinity chromatography material, and/or a cation-exchange (CEX) chromatography, wherein optionally the affinity chromatography material can be a chromatography material coupled to an OX21 monoclonal antibody.

In certain embodiments, the method comprises contacting a preparation containing precursor and mature form complement factor I proteins with a chromatography material coupled to an OX21 monoclonal antibody.

In certain embodiments, method comprises the following steps;
(a) contacting a preparation comprising a mixture of a precursor complement factor I protein and a mature form complement factor I protein with a cation-exchange chromatography material under conditions that said precursor complement factor I protein and said mature form complement factor I protein each bind to the chromatography material;
(b) contacting the chromatography material with one or more salt containing elution buffer solutions; and
(c) eluting said precursor complement factor I protein and said mature form complement factor I protein; in order to obtain a series of distinct eluates,

wherein, within the series of eluates, the precursor complement factor I protein and mature form complement factor I protein are substantially separated from one another.

In certain embodiments, prior to step (a) the method further comprises;
i) contacting the preparation comprising a mixture of a precursor complement factor I protein, a mature form complement factor I protein and one or more cellular components with an affinity chromatography material under conditions that enable said precursor complement factor I protein and said mature form complement factor I protein to each bind to the chromatography material;
ii) contacting the chromatographic material with one or more salt containing elution buffer solutions; and
iii) eluting said precursor complement factor I protein and said mature form complement factor I protein in order to obtain a second preparation of said precursor complement factor I protein and said mature form complement factor I protein which is substantially free of the one or more cellular components.

In certain embodiments, the method involves substantially separating mature form complement factor I and/or precursor complement factor I from one or more cellular components. For example, substantially separating mature form complement factor I and/or precursor complement factor I from a cell culture.

In certain embodiments, the method involves substantially separating mature form complement factor I and/or precursor complement factor I from one or more cellular components by contacting a preparation containing mature form complement factor I and/or precursor complement factor I and one or more cellular components with an affinity chromatography material. In certain embodiments, the method involves substantially separating mature form complement factor I and/or precursor complement factor I from one or more cellular components by contacting a preparation containing mature form complement factor I and/or precursor complement factor I and one or more cellular components with a chromatography material coupled to an OX21 monoclonal antibody.

In certain embodiments, the method involves eluting precursor complement factor I protein and/or mature form complement factor I protein in order to obtain a preparation of said precursor complement factor I protein and/or mature form complement factor I protein which is substantially free of one or more cellular components.

In certain embodiments, the method involves substantially separating mature form complement factor I and/or precursor complement factor I from one or more cellular components before contacting a preparation comprising a mixture of a precursor complement factor I protein and a mature form complement factor I protein with a cation-exchange chromatography material.

In some embodiments, the method involves contacting precursor and mature form complement factor I protein bound to chromatographically active material with one or more elution buffers wherein a buffer (i.e. an aqueous solution resistant to pH change by the action of its acid-base conjugate components) is passed over chromatography material onto which precursor and mature form CFI protein is bound.

In some embodiments, the method involves eluting precursor and mature from complement factor I protein from a chromatography material, in order to obtain distinct eluates containing predominantly precursor or mature form complement system proteins, wherein aqueous buffer is passed over the chromatography material to which the precursor and mature form complement factor I proteins are each bound to affect dissociation of the mature form or precursor from the chromatography material into said distinct eluates of aqueous buffer.

In some embodiments, the method involves contacting precursor and mature complement factor I protein bound to chromatographically active material with one or more wash buffers wherein a buffer (i.e. an aqueous solution resistant to pH change by the action of its acid-base conjugate components) is passed over chromatography material onto which precursor and mature complement factor I protein is bound.

In certain embodiments the complement factor I protein is mammalian CFI, e.g. a human CFI protein.

The term column volume (CV) refers to the volume of chromatography column or cartridge that is not occupied by the media.

In some embodiments, the method involves steps wherein total complement factor I protein is bound to the resin at a dynamic binding capacity (DBC) of about 50% to 90% (e.g. 50%, e.g. 60%, e.g. 70% e.g. 80%, e.g. 90%). In some embodiments, the method involves steps wherein the total complement factor I protein are bound to the resin at a dynamic binding capacity (DPC) of about 50% to 99% (e.g. 50%, e.g. 60%, e.g. 70% e.g. 80%, e.g. 90% e.g. 95%, e.g. 99%).

The term “dynamic binding capacity” (DBC) defines the amount of product that will bind to the chromatographically active material under typical flow conditions and must be determined under relevant flow conditions and load characteristics. It is calculated based on the amount that can be loaded before significant product levels are measured in the flow through (the breakthrough point)

In some embodiments, the method involves steps wherein the eluate is neutralized to a pH of about 7.0 to 8.2 e.g. the pH of the eluate may be adjusted to value of 6.5, 6.6, 6.7, 6.8, 6.9 7.0, 7.1, 7.2, 7.3, 7.4, 7.5, 7.6, 7.7, 7.8, 7.9, 8.0, 8.1 or 8.2)

Throughout the description and claims of this specification, the words “comprise” and “contain” and variations of them mean “including but not limited to” and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of the features and/or steps are mutually exclusive.

The invention is not restricted to any details of any foregoing embodiments. The invention extends to any novel one, or novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

### Example 1—Evaluation of Fluid Phase Cofactor Activity of Pro-CFI

Materials and Methods & Results

Assessment of the cleavage ability of Pro-CFI was determined by a fluid phase cofactor assay. 1 μg of C3b, 250ng of Factor H and 10ng of Pro-CFI was mixed in a final volume of 15 μL Phosphate Buffer Saline (PBS) (9.5 mM PO₄ without Calcium or Magnesium), and incubated at 37° C. for 1.5 hours. The reaction was stopped by the addition of reducing laemmli buffer at 5, 15, 30, and 45-minute intervals (2X Laemmli Sample Buffer (Bio-Rad,161-0737): 65.8 mM Tris-HCl, pH 6.8, 26.3% (w/v) glycerol,2.1% SDS, 0.01% bromophenol blue. To make reducing −50 μl of 2-β-mercaptoethanol was added to 950 μl sample buffers. Samples were mixed 1:1 with the Laemmli Buffer). Proteolytic cleavage of C3b was determined using a 10% SDS-PAGE gel stained with Coomassie.

### Example 2—Expression Vector for Solely Recombinant Production of Mature CFI

Materials and Methods

Vector Design

To address the issue of mature CFI and Pro-CFI co-production, a CFI expression vector was designed (VB171219-1127wqz) (FIG. 6). The vector contains an internal ribosomal entry site (IRES) to facilitate the production of two proteins (pro-CFI and furin) from a single polycistronic mRNA through cap-independent translation. Furin was positioned upstream of the IRES element to ensure excess Furin relative to Pro-CFI (the upstream transcript is typically produced in higher quantities than the downstream transcript). The vector also contains an SV40 domain which enhances protein expression in mammalian cell lines. Two antibiotic resistance genes (ampicillin and hygromycin) were included to facilitate selection.

Transfection

The vector DNA was amplified using a maxiprep kit (QIAGEN, Cat No./ID: 12163) and sequence verified prior to transfection into human embryonic kidney 293T (ATCC® CRL-3216™) cells. JetPEI reagent (Polyplus-transfection; 101-40N) was used for the transfections following the manufacturer's protocol. HEK293T were cultured in DMEM medium (Gibco, 11966025) supplemented with 10% heat-inactivated Foetal Bovine Serum (Gibco, 10270106), 6.06 mM L-glutamine solution (Gibco, 25030081) and 100 U/mL Penicillin-Streptomycin solution (Gibco, 15140122) at 37° C. and 5% CO₂. Stable transfected cells were selected in the presence of 0.4 mg/ml Hygromycin B (Sigma-Aldrich, H3274). Single clones were identified by limiting dilution in the presence of a feeder layer of wild-type HEK293T cells, and the highest expressors selected for protein production. Cells were again cultured in multilayer culture flasks (Millicell HY 5-layer, Millipore) in the same media as above for 10 days at 37° C. and 5% CO₂ before harvesting the supernatant. HEK293T cells are adherent, so the supernatant was removed by pipetting using a stripette pipette. Once the supernatant was collected, it was spun in a centrifuge at 3600 g to pellet any cell debris prior to filtering using a 0.22 μm pore filter (GPWPO4700).

Factor I Purification

An OX21 column was generated by coupling OX21 antibody (Public Health England, 91060417 ECACC) to a 1 ml HiTrap NHS-activated HP column (GE Healthcare, 17071601) according to the manufacturer's instructions (GE Healthcare—Instructions 71-7006-00, 2014).

Factor I purification was performed using an AKTA Start protein purification system (GE Healthcare, 29022094-ECOMINSSW). First, the system was primed with running buffer (PBS) before attaching the OX21 column. The supernatant was loaded onto the column, and unbound protein removed using running buffer. The bound CFI was then eluted with 0.1M Glycine (pH 2.7) into 1 ml fractions at a flow rate of 1 ml per minute. 1M Tris-base (pH 9.0) was added to neutralise the pH, before buffer exchange into PBS using a PD-10 desalting column (GE Healthcare, 17085101).

Silver Staining

Purified Factor I was visualised on a 12% SDS-PAGE gel following silver staining. Firstly, the protein was quantified using a NanoDrop™ One^{c} Microvolume UV-Vis Spectrophotometer using the cuvette. 70 μl sample was loaded into the cuvette. The A₂₈₀ reading was multiplied by (Molecular Weight (kDa)/Extinction Coefficient (M-1 cm-1)) to calculate the concentration (mg/ml). 1 μg of protein was loaded into each well. The silver stain was achieved by first soaking the gel in 50% (v/v) methanol, 10% (v/v) acetic acid for 30 minutes prior to another 30 minutes in 5% (v/v) methanol, 7% (v/v) acetic acid. The acetic acid and methanol mixture were then washed off before a final 30-minute incubation in 5% glutaraldehyde, and an overnight wash in deionised water. The gel was then immersed in a 5 μg/ml dithiothreitol solution for 30 minutes before staining with 0.1% (w/v) silver nitrate. The stain was developed using a solution of 0.28M sodium carbonate and 0.0185% (v/v) formaldehyde until brown bands formed. To quench the reaction, citric acid (Sigma, 251275) was added after 10 minutes.

Comparison of Fluid Phase Cofactor Activity Between Serum Purified FI and IRES-Vector FI

To determine the complement regulatory activity of IRES-vector generated FI compared to serum purified CFI, a fluid phase co-factor assay was performed. Briefly, 1 pg C3b and 250ng Factor H were mixed with either 10 ng recombinant CFI or serum purified CFI, to a total volume of 15 μl in PBS. The reaction mixtures were incubated at 37° C. for 1 hour, and separate aliquots were removed and treated with laemmli buffer contain 2-β-mercaptoethanol at 5, 15, 30, 45, and 60-minute intervals. Assessment of the proteolytic breakdown of C3b was achieved using a 10% SDS-PAGE gel which had been stained with Coomassie.

Results

Recombinant Factor I Generated Using Invention Compared to CFI from Serum

To assess the ability of the present invention to produce fully processed CFI without post-purification enzymatic treatment with Furin, silver staining of the purified CFI was performed. Silver stain of SDS page comparing serum purified Factor I against IRES vector produced CFI under reducing conditions there is a single band at 88 kDa for both IRES CFI and serum CFI. Under reducing conditions there are two bands present for both IRES CFI and serum CFI at 50 kDa and 38 kDa corresponding to the heavy and light chains, respectively. The result demonstrated that fully processed CFI with no contaminating Pro-CFI could be obtained using the invention.

Function of Recombinant CFI Generated Using Embodiments of the Present Invention

To assess the functional activity of CFI generated using the invention, serum purified CFI (Complement Technology, A138) and purified recombinant CFI were compared in cofactor assays. Comparison of fluid phase cofactor activity between serum purified FI IRES-vector FI. C3b, Factor H, and 10ng of either IRES CFI or serum purified FI were incubated in solution at 37° C. for 1 hour. The reaction was stopped by the addition of reducing laemmli buffer at 5, 15, 10, 45 and 60 minute intervals. C3b breakdown was assessed by SDS-PAGE and Coomassie staining. A decrease of the C3α′ (110 kDa) and the appearances of several other C3α′ bands (68 kDa, 46 kDa and 43 kDa) indicative of inactivation of C3b through proteolytic cleavage.

Enzymatic activity of IRES CFI and serum CFI was demonstrated to be equivilant.

### Example 3—Separation and Isolation of Pro-CFI and Mature CFI

Materials and Methods

Construct Design:

A modified pDR2 EF1α (pDEF-CFI) expression vector with an inserted mammalian CFI sequence (CFI Accession: Y00318 M25615; VERSION Y00318.1) was used for the recombinant production of pro-CFI.

Transfection:

The vector DNA was amplified using a maxiprep kit (QIAGEN, Cat No./ID: 12163) and the sequence verified prior to transfection into CHO-K1 (ATCC® CCL-61™) cells using jetPEI (Polyplus; VWR, Leicestershire, UK) following the manufacture's protocols. Transfected CHO were cultured in DMEM-F12 (Gibco, 11320033) supplemented with 10% heat-inactivated Fetal Bovine Serum (Gibco) and 5 mL Penicillin-Streptomycin-Glutamine (Gibco, 10378016) at 37° C. with 5% CO₂. Stable transfected cells were selected in the presence of 0.6 mg/ml Hygromycin B. Single clones were generated by limiting dilution and the highest expressors selected for protein production. Cells were cultured in roller bottles for 10 days when the media was changed, before harvesting the supernatant on the 14^{th} day CHO cells are adherent, so the supernatant was removed by pipetting using a stripette pipette. Once the supernatant was collected, it was spun in a centrifuge at 3600 g to pellet any cell debris prior to filtering using a 0.22 μm pore filter (GPWPO4700). A mixture of mature Factor I and Pro-I (approximately 20-30% pro-CFI) was purified from the cell supernatant using a 1 ml HiTrap NHS activated HP column (GE Healthcare) coupled with OX-21 monoclonal antibody.

Dialysis:

The purified product was collated and transferred to 3.5 KDa dialysis tubing (Thermo Scientific, 10005743), before dialysing at 4° C. overnight in 50 mM Sodium phosphate monobasic (Sigma-Aldrich, S8282), pH 6.

Cation Exchange Chromatography:

To facilitate loading on a Mono S 5/50 GL column (Sigma-Aldrich, GE17-5168-01), the purified proteins were concentrated using a Vivaspin 30,000 kDa molecular weight cut-off centrifugal concentrator (Z614637, Sigma). First the column was equilibrated with 5 column volumes (CV) of the dialysis buffer (50 mM Sodium phosphate monobasic, pH 6) at 1.5 ml/min, before injecting the preparation using a 10 mL Superloop. Once the preparation was injected, the column was washed with 2 CV of dialysis buffer before eluting using a linear salt gradient. The percentage of the salt buffer (50 mM Sodium phosphate monobasic, pH 6, 1M NaCl) increased from 0% to 40% over 30 CV (30 mL). Pro-CFI and CFI have different isoelectric points, and thus elute at different salt concentrations. The eluted proteins were collected in a 96 deep well plate in 0.5 ml fractions, and samples from the resulting fractions run on SDS PAGE and stained with Coomassie Blue. The fractions from the first peak contain fully mature CFI and the fractions from the second peak contain Pro-CFI (FIG. 10). Either mature CFI or Pro-CFI were collated, and buffer exchanged into PBS using a PD10 desalting column (Cytiva, 17085101) before freezing.

Results

Visualisation of Pro-CFI generated:

To determine whether the method of certain embodiments could produce Pro-CFI, an SDS PAGE stained with Coomassie Blue and a Western blot were performed. Samples were first run on SDS PAGE and transferred to a nitrocellulose membrane using a transfer tank, according to the manufacturer's protocols (Bio-Rad). The transfer tank was filled with Transfer buffer (25 mM Tris, 190 mM glycine, 20% Methanol, in distilled water) and the blotting cassette was assembled. The cassette consisted of: two sponges, two pieces of filter paper, one piece of nitrocellulose and one SDS PAGE gel. The cassette was then slotted into the tank and the samples were transferred for 90 minutes at 100 V and 400 mA using a PowerPac (Bio-Rad). The membrane was then blocked with 5% skimmed milk in PBST (0.1% Tween 20) for 1 hour at room temperature. A roller shakerwas used to ensure complete coverage of the membrane. The membrane was then incubated with a sheep polyclonal antibody to Factor I (Abcam, ab8843) (1 pg/ml) in blocking buffer. The blot was incubated at 4° C. overnight. The membrane was washed three times with 0.1% PBST and incubated for 1 hour at room temperature with donkey anti sheep IgG-horse radish peroxidase (Jackson ImmunoResearch, 713-035-147) diluted 1:3000 in blocking buffer. The membrane was washed again and developed using Clarity Western ECL substrate (BioRad, 1705060) according to the manufacturer's protocols. The Blot was then visualised on the Odyssesy Fc Imaging system (Licor). Under reducing conditions, the fractions collected from the first elution peak produced bands corresponding to the heavy and light chains of mature CFI, and the fractions from the second peak generated a band corresponding to Pro-CFI only. For the peak fractions there was no evidence of cross-contamination of either species.

The invention is further described by the following numbered paragraphs:
1. An expression system for producing a recombinant mature Complement Factor I protein or variant thereof, the system comprising:
   a. a nucleic acid molecule encoding a recombinant precursor Complement Factor I protein or variant thereof; and
   b. a nucleic acid molecule encoding a furin protein or variant thereof wherein said furin protein or variant thereof is capable of cleaving the encoded precursor Complement Factor I protein to produce a recombinant mature Complement Factor I protein, wherein optionally the furin protein is capable of cleaving greater than 50% of the encoded precursor Complement Factor I protein.
2. An expression system according to paragraph 1, which comprises an expression vector which comprises the nucleic acid molecule encoding the precursor Complement Factor I protein or variant thereof and the nucleic acid molecule encoding the furin protein or variant thereof.
3. An expression system according to paragraph 1 or paragraph 2, wherein the expression vector comprises a promoter element.
4. An expression system according to paragraph 3, wherein the promoter element is upstream of the nucleic acid molecule encoding a precursor complement Factor I protein or variant thereof
5. An expression system according to paragraph 3 or paragraph 4 wherein the promoter element is upstream of the nucleic acid molecule encoding a furin protein.
6. An expression system according to any preceding paragraph, wherein the expression vector comprises a nucleic acid molecule encoding a translation initiation sequence.
7. An expression system according to paragraph 8, wherein the nucleic acid molecule encoding a translation initiation sequence is positioned downstream of the promoter element and upstream of the nucleic acid molecule encoding the precursor Complement Factor I protein or variant thereof and the nucleic acid molecule encoding the furin protein or variant thereof.
8. An expression system according to any of paragraphs 2 to 7, wherein the expression vector further comprises a nucleic acid molecule encoding an internal ribosome entry site.
9. An expression system according to paragraph 8, wherein the nucleic acid molecule encoding the internal ribosome entry site comprises a nucleic acid sequence as set forth in SEQ. ID. 9.
10. An expression system according to any preceding paragraph, wherein the recombinant mature human Complement Factor I protein comprises a first amino acid sequence selected from an amino acid sequence as set forth in SEQ. ID. No. 2 (heavy chain) or an amino acid sequence having at least 85% sequence identity to the amino acid sequence as set forth in SEQ. ID. No. 2, and a second amino acid sequence as set forth in SEQ. ID. 3 (light chain) or an amino acid sequence having at least 85% sequence identity to the amino acid sequence set forth in SEQ. ID. No. 3, wherein the first and second amino acid sequences are linked via a disulphide bond.
11. An expression system according to any preceding paragraph, wherein the recombinant precursor human Complement Factor I comprises an amino acid sequence as set forth in SEQ. ID. No. 1, or an amino acid sequence having at least 90% sequence identity to the amino acid sequence as set forth in SEQ. ID. No. 1, e.g. 91, 92, 93, 94, 95, 96, 97, 98 or 99% sequence identity.
12. An expression system according to any preceding paragraph, wherein the furin protein comprises an amino acid sequence selected from an amino acid sequence as set forth in SEQ. ID. No. 5 or an amino acid sequence having at least 85% sequence identity to the amino acid sequence set forth in SEQ. ID. No 5.
13. An expression system according to paragraph 12, wherein the furin protein comprises an amino acid sequence having at least 90% sequence identity to the amino acid sequence as set forth in SEQ. ID. No: 5, e.g. 91, 92, 93, 94, 95, 96, 97, 98 or 99% sequence identity.
14. An expression system according to any preceding paragraph, wherein the vector comprises a nucleic acid sequence selected from the nucleic acid sequence as set forth in SEQ. ID. No. 10 or a nucleic acid sequence having at least 85% sequence identity to the nucleic acid sequence as set forth in SEQ. ID. No. 10.
15. An expression system according to any of paragraphs 2 to 14, wherein the vector comprises a nucleic acid sequence having at least 90% sequence identity to the nucleic acid sequence as set forth in SEQ. ID. No. 8, e.g. 91, 92, 93, 94, 95, 96, 97, 98 or 99% sequence identity.
16. An expression system according to any of paragraphs 2 to 15, wherein the expression vector comprises at least one nucleic acid molecule encoding a resistance marker.
17. An expression system according to any preceding paragraph for use as a medicament.
18. An expression system according to paragraph 17 for use to treat a complement system mediated disorder.
19. An expression system for use according to paragraph 18, wherein the complement system mediated disorder is selected from atypical haemolytic uremic syndrome, microangiopathic hemolytic anaemia, age-related macular degeneration, C3 Glomerulopathy, Alzheimer's disease, cerebral inflammation and/or thrombocytopenia.
20. An expression system for use according to any of paragraphs 17 to 19 wherein the expression vector is a viral vector.
21. A method for producing a recombinant mature Complement Factor I protein or variant thereof, the method comprising:
   a. expressing a recombinant precursor Complement Factor I protein or variant thereof and a recombinant furin protein or variant thereof in a host cell under conditions suitable for the expressed furin protein to cleave the expressed recombinant precursor Complement Factor I protein or variant thereof to form the recombinant mature Complement Factor I protein or variant thereof, wherein optionally greater than 50% of the recombinant precursor complement factor I protein is cleaved by the furin protein.
22. A method according to paragraph 21, which comprises:
   a. transfecting a host cell with an expression vector which comprises a nucleic acid molecule encoding the precursor Complement Factor I protein or variant thereof and a nucleic acid molecule encoding the furin protein.
23. A method according to paragraph 22, wherein the expression vector further comprises:
   a. a nucleic acid molecule encoding an internal ribosome entry site, wherein the nucleic acid molecule encoding the internal ribosome entry site is positioned between the nucleic acid molecule encoding the precursor Complement Factor I protein or variant thereof and the nucleic acid molecule encoding the furin protein;
   b. a promoter element, wherein the promoter element is positioned upstream of the nucleic acid molecule encoding the precursor Complement Factor I protein or variant thereof and the nucleic acid molecule encoding the furin protein;
   c. a translation initiation sequence, wherein the translation initiation sequence is positioned downstream of the promoter element and upstream of the nucleic acid molecule encoding the precursor Complement Factor I protein or variant thereof and the nucleic acid molecule encoding the furin protein.
24. A method according to any of paragraphs 22 to 23 which comprises expressing the nucleic acid molecule encoding a recombinant precursor Complement Factor I protein or variant thereof and the nucleic acid molecule encoding a recombinant furin protein in a eukaryotic cell, wherein optionally the eukaryotic cell is a Human Embryonic Kidney 293T cell.
25. A method according to any of paragraphs 22 to 24, which comprises expressing the recombinant precursor Complement Factor I protein and a recombinant furin protein in an expression system according to any of paragraphs 1 to 18.
26. A method according to any of paragraphs 22 to 25, which further comprises recovering the recombinant mature Complement Factor I protein.
27. A mature recombinant Complement Factor I protein obtainable by a method according to any of paragraphs 22 to 26.
28. A therapeutic composition comprising a mature recombinant Complement Factor 1 according to paragraph 27.
29. A therapeutic composition according to paragraph 28 for use in the treatment of a subject in need thereof.
30. A therapeutic composition according to paragraph 29 for use in the treatment of a subject suffering from a disorder selected from atypical hemolytic uremic syndrome, microangiopathic hemolytic anemia, age-related macular degeneration, C3 Glomerulopathy, Alzheimer's disease, cerebral inflammation and/or thrombocytopenia.
31. A method for separating a recombinant mature complement factor I (CFI) protein from one or more cellular components wherein the method comprises the following steps;
   (a) contacting a preparation comprising a mixture of a precursor complement factor I protein, a mature complement factor I protein and one or more further cell components with a chromatographic material under conditions that enable said precursor complement factor I protein and said mature form complement factor I protein to each bind to the chromatographic material;
   (b) contacting the chromatographic material with one or more salt containing elution buffer solutions; and
   (c) eluting said precursor complement factor I protein and mature complement factor I protein to obtain a series of eluates, wherein, within the series of eluates, the precursor complement system protein and mature form complement system protein are substantially separated from one another, and/or wherein the precursor complement factor I protein and mature form complement factor I protein are substantially separated from other cellular components, wherein optionally the chromatographic material comprises an anti-OX21 antibody.
32. The method according to paragraph 31, which further comprises the following steps;
   d) contacting a preparation comprising the eluates comprising mature Complement Factor I protein and precursor Complement Factor I protein with at least one further chromatographic material under conditions that said precursor Complement Factor I protein and said mature form Complement Factor I protein bind to the at least one further chromatographic material;
   e) contacting the at least one further chromatographic material with one or more salt containing elution buffer solutions; and
   f) eluting said precursor complement system protein and mature complement system protein; in order to obtain a further series of distinct eluates,
wherein, within the further series of eluates, the precursor complement factor I protein and mature form complement factor I protein are substantially separated from one another, wherein optionally the further chromatography material is a cation-exchange (CEX) chromatography material.
33. The method according to paragraph 31, wherein the elution buffer solutions contacting the cation exchange chromatography material have a pH of about 4.5-7.5, optionally about pH 6.0 and/or wherein the elution buffer solutions contacting the affinity chromatography material have a pH of about 1.5-4.5, optionally about pH 2.7.
34. The method according to paragraph 33, wherein the elution buffer solutions contacting the affinity chromatography material comprise glycine, wherein optionally the glycine is at a concentration of 0.1M and optionally wherein the precursor Complement Factor I protein and the mature Complement Factor I protein are present in the preparation at a molar ratio of about 2.5:7.5.

1. An expression vector for producing a recombinant mature Complement Factor I protein or variant thereof, the expression vector comprising:
a. a nucleic acid molecule encoding a recombinant precursor Complement Factor I protein or variant thereof; and
b. a nucleic acid molecule encoding a furin protein or variant thereof wherein said furin protein or variant thereof is capable of cleaving the encoded precursor Complement Factor I protein to produce a recombinant mature Complement Factor I protein, wherein optionally the furin protein or variant thereof is capable of cleaving greater than 50% of the encoded precursor Complement Factor I protein.

2. An expression system comprising the expression vector of claim 1.

3. An expression vector or system according to claim 1 or claim 2, wherein the expression vector comprises a promoter element.

4. An expression vector or system according to claim 3, wherein the promoter element is upstream of the nucleic acid molecule encoding a precursor complement Factor I protein or variant thereof

5. An expression vector or system according to claim 3 or claim 4 wherein the promoter element is upstream of the nucleic acid molecule encoding a furin protein or variant thereof.

6. An expression vector or system according to any preceding claim, wherein the expression vector comprises a nucleic acid molecule encoding a translation initiation sequence.

7. An expression vector or system according to claim 6, wherein the nucleic acid molecule encoding a translation initiation sequence is positioned downstream of the promoter element and upstream of the nucleic acid molecule encoding the precursor Complement Factor I protein or variant thereof and the nucleic acid molecule encoding the furin protein or variant thereof.

8. An expression vector or system according to any preceding claim, wherein the expression vector further comprises a nucleic acid molecule encoding an internal ribosome entry site (IRES), preferably wherein the nucleic acid molecule encoding an IRES is positioned between the nucleic acid molecule encoding the precursor Complement Factor I protein or variant thereof and the nucleic acid molecule encoding the furin protein or variant thereof.

9. An expression vector or system according to claim 8, wherein the nucleic acid molecule encoding the internal ribosome entry site comprises a nucleic acid sequence as set forth in SEQ. ID. No. 9.

10. An expression vector or system according to any preceding claim, wherein the recombinant mature human Complement Factor I protein comprises a first amino acid sequence selected from an amino acid sequence as set forth in SEQ. ID. No.2 (heavy chain) or an amino acid sequence having at least 85% sequence identity to the amino acid sequence as set forth in SEQ. ID. No. 2, and a second amino acid sequence as set forth in SEQ. ID. No. 3 (light chain) or an amino acid sequence having at least 85% sequence identity to the amino acid sequence set forth in SEQ. ID. No. 3, wherein the first and second amino acid sequences are linked via a disulphide bond.

11. An expression vector or system according to any preceding claim, wherein the recombinant precursor human Complement Factor I comprises an amino acid sequence as set forth in SEQ. ID. No. 1, or an amino acid sequence having at least 90% sequence identity to the amino acid sequence as set forth in SEQ. ID. No. 1, e.g. 91, 92, 93, 94, 95, 96, 97, 98 or 99% sequence identity.

12. An expression vector or system according to any preceding claim, wherein the furin protein comprises an amino acid sequence selected from an amino acid sequence as set forth in SEQ. ID. No. 5 or an amino acid sequence having at least 85% sequence identity to the amino acid sequence set forth in SEQ. ID. No 5.

13. An expression vector or system according to claim 12, wherein the furin protein comprises an amino acid sequence having at least 90% sequence identity to the amino acid sequence as set forth in SEQ. ID. No: 5, e.g. 91, 92, 93, 94, 95, 96, 97, 98 or 99% sequence identity.

14. An expression vector or system according to any preceding claim, wherein the vector comprises a nucleic acid sequence selected from the nucleic acid sequence as set forth in SEQ. ID. No. 10 or a nucleic acid sequence having at least 85% sequence identity to the nucleic acid sequence as set forth in SEQ. ID. No. 10.

15. An expression vector or system according to any of claims 1 to 14, wherein the vector comprises a nucleic acid sequence having at least 90% sequence identity to the nucleic acid sequence as set forth in SEQ. ID. No. 8, e.g. 91, 92, 93, 94, 95, 96, 97, 98 or 99% sequence identity.

16. An expression vector or system according to any of claims 1 to 15, wherein the expression vector comprises at least one nucleic acid molecule encoding a resistance marker.

17. An expression system for producing a recombinant mature Complement Factor I protein or variant thereof, the expression system comprising:
a. a nucleic acid molecule encoding a recombinant precursor Complement Factor I protein or variant thereof; and
b. a nucleic acid molecule encoding a furin protein or variant thereof wherein said furin protein or variant thereof is capable of cleaving the encoded precursor Complement Factor I protein to produce a recombinant mature Complement Factor I protein, wherein optionally the furin protein or variant thereof is capable of cleaving greater than 80%, 85%, 90% or 95% of the encoded precursor Complement Factor I protein.

18. An expression vector or system according to any preceding claim for use as a medicament.

19. An expression vector or system according to any of claims 1 to 17 for use in treating a complement system mediated disorder.

20. An expression vector or system for use according to claim 19, wherein the complement system mediated disorder is selected from atypical haemolytic uremic syndrome, microangiopathic hemolytic anaemia, age-related macular degeneration, C3 Glomerulopathy, Alzheimer's disease, cerebral inflammation and/or thrombocytopenia, or an ocular complement related disease or condition such as age related macular degeneration (AMD), glaucoma, diabetic retinopathy, early-onset macular degeneration, Stargardt's disease, central serous chorioretinopathy, retinitis pigmentosa or uveitis, preferably AMD.

21. An expression system for use according to any of claims 18 to 20 wherein the expression vector is a viral vector, preferably an adeno-associated virus (AAV) vector.

22. A method of treating a complement system mediated disorder in a subject, the method comprising administering the expression vector or system according to any of claims 1 to 17, preferably wherein the method comprises administering the expression vector or system to a region of an eye of a subject.

23. An adeno-associated viral (AAV) vector particle comprising
a. a nucleic acid sequence encoding a precursor Complement Factor I protein or variant thereof; and
b. a nucleic acid sequence encoding a furin protein or variant thereof wherein said furin protein or variant thereof is capable of cleaving the encoded precursor Complement Factor I protein to produce a recombinant mature Complement Factor I protein.

24. The AAV vector particle according to claim 23, wherein the vector particle further comprises a promoter.

25. The AAV vector particle according to claim 23 or 24 wherein the vector particle further comprises a nucleic acid sequence encoding an IRES, preferably wherein the nucleic acid sequence encoding an IRES is positioned between the nucleic acid molecule encoding the precursor Complement Factor I protein or variant thereof and the nucleic acid molecule encoding the furin protein or variant thereof.

26. The AAV vector particle according to any of claims 23 to 25, wherein the AAV vector particle comprises an AAV2 genome and AAV2 capsid proteins, an AAV2 genome and AAV5 capsid proteins, or an AAV2 genome and AAV8 capsid proteins.

27. A pharmaceutical composition comprising the AAV vector particle according to any of claims 23 to 26.

28. The AAV vector particle or composition according to any of claims 23-27 for use as a medicament, preferably for use in treating a complement system mediated disorder

29. The AAV vector particle or composition for use according to claim 28, wherein the complement system mediated disorder is selected from atypical haemolytic uremic syndrome, microangiopathic hemolytic anaemia, age-related macular degeneration, C3 Glomerulopathy, Alzheimer's disease, cerebral inflammation and/or thrombocytopenia, or an ocular complement related disease or condition such as age related macular degeneration (AMD), glaucoma, diabetic retinopathy, early-onset macular degeneration, Stargardt's disease, central serous chorioretinopathy, retinitis pigmentosa or uveitis, preferably AMD

30. A method for producing a recombinant mature Complement Factor I protein or variant thereof, the method comprising:
a. expressing a recombinant precursor Complement Factor I protein or variant thereof and a recombinant furin protein or variant thereof in a host cell under conditions suitable for the expressed furin protein or variant thereof to cleave the expressed recombinant precursor Complement Factor I protein or variant thereof to form the recombinant mature Complement Factor I protein or variant thereof, wherein optionally greater than 80% of the recombinant precursor complement factor I protein or variant thereof is cleaved by the furin protein or variant thereof.

31. A method according to claim 30, which comprises:
a. transfecting a host cell with an expression vector which comprises a nucleic acid molecule encoding the precursor Complement Factor 1 protein or variant thereof and a nucleic acid molecule encoding the furin protein or variant thereof.

32. A method according to claim 31, wherein the expression vector further comprises:
a. a nucleic acid molecule encoding an internal ribosome entry site, wherein the nucleic acid molecule encoding the internal ribosome entry site is positioned between the nucleic acid molecule encoding the precursor Complement Factor I protein or variant thereof and the nucleic acid molecule encoding the furin protein or variant thereof;
b. a promoter element, wherein the promoter element is positioned upstream of the nucleic acid molecule encoding the precursor Complement Factor I protein or variant thereof and the nucleic acid molecule encoding the furin protein or variant thereof;
c. a translation initiation sequence, wherein the translation initiation sequence is positioned downstream of the promoter element and upstream of the nucleic acid molecule encoding the precursor Complement Factor I protein or variant thereof and the nucleic acid molecule encoding the furin protein or variant thereof.

33. A method according to any of claims 31 to 32 which comprises expressing the nucleic acid molecule encoding a recombinant precursor Complement Factor 1 protein or variant thereof and the nucleic acid molecule encoding a recombinant furin protein or variant thereof in a eukaryotic cell, wherein optionally the eukaryotic cell is a Human Embryonic Kidney 293T cell.

34. A method according to any of claims 31 to 33, which comprises expressing the recombinant precursor Complement Factor I protein or variant thereof and a recombinant furin protein or variant thereof in an expression vector or system according to any of claims 1 to 17.

35. A method according to any of claims 31 to 34, which further comprises recovering the recombinant mature Complement Factor I protein or variant thereof.

36. A mature recombinant Complement Factor I protein or variant thereof obtainable by a method according to any of claims 31 to 35.

37. A therapeutic composition comprising a mature recombinant Complement Factor I or variant thereof according to claim 36.

38. A therapeutic composition according to claim 37 for use in the treatment of a subject in need thereof, preferably for use in the treatment of a subject suffering from a disorder such as atypical hemolytic uremic syndrome, microangiopathic hemolytic anemia, C3 Glomerulopathy, Alzheimer's disease, cerebral inflammation, thrombocytopenia, or an ocular complement related disease or condition such as age related macular degeneration (AMD), glaucoma, diabetic retinopathy, early-onset macular degeneration, Stargardt's disease, central serous chorioretinopathy, retinitis pigmentosa or uveitis, preferably AMD.

39. A method for separating a recombinant mature complement factor I (CFI) protein from one or more cellular components wherein the method comprises the following steps;
(a) contacting a preparation comprising a mixture of a precursor complement factor I protein, a mature complement factor I protein and one or more further cell components with a chromatographic material under conditions that enable said precursor complement factor I protein and said mature form complement factor I protein to each bind to the chromatographic material;
(b) contacting the chromatographic material with one or more salt containing elution buffer solutions; and
(c) eluting said precursor complement factor I protein and mature complement factor I protein to obtain a series of eluates, wherein, within the series of eluates, the precursor complement system protein and mature form complement system protein are substantially separated from one another, and/or wherein the precursor complement factor I protein and mature form complement factor I protein are substantially separated from other cellular components, wherein optionally the chromatographic material comprises an anti-OX21 antibody.

40. The method according to claim 39, which further comprises the following steps;
d) contacting a preparation comprising the eluates comprising mature Complement Factor I protein and precursor Complement Factor I protein with at least one further chromatographic material under conditions that said precursor Complement Factor I protein and said mature form Complement Factor I protein bind to the at least one further chromatographic material;
e) contacting the at least one further chromatographic material with one or more salt containing elution buffer solutions; and
f) eluting said precursor complement system protein and mature complement system protein; in order to obtain a further series of distinct eluates,
wherein, within the further series of eluates, the precursor complement factor I protein and mature form complement factor I protein are substantially separated from one another, wherein optionally the further chromatography material is a cation-exchange (CEX) chromatography material.

41. The method according to claim 39, wherein the elution buffer solutions contacting the cation exchange chromatography material have a pH of about 4.5-7.5, optionally about pH 6.0 and/or wherein the elution buffer solutions contacting the affinity chromatography material have a pH of about 1.5-4.5, optionally about pH 2.7.

42. The method according to claim 41, wherein the elution buffer solutions contacting the affinity chromatography material comprise glycine, wherein optionally the glycine is at a concentration of 0.1M and optionally wherein the precursor Complement Factor I protein and the mature Complement Factor I protein are present in the preparation at a molar ratio of about 2.5:7.5.
